(19) United States
(12) Patent Application Publication
(10) Pub. No.: US 2024/0108210 A1
(43) Pub. Date: Apr. 4, 2024

(54) Imaging Guidewire Having Telescopically Movable Imaging Core

(71) Applicant: CANON U.S.A., INC., MELVILLE, NY (US)

(72) Inventor: Mark Alan Hamm, Lynnfield, MA (US)

(21) Appl. No.: 17/937,124

(22) Filed: Sep. 30, 2022

(51) Int. Cl. A61B 1/313; A61B 1/00; A61B 1/07; A61B 5/00

(52) U.S. Cl. CPC A61B 1/3137; A61B 1/00096; A61B 1/00154; A61B 1/07; A61B 5/6851; A61M 2025/0042

(57) ABSTRACT

An imaging guidewire comprises a first hypotube assembly that forms a guidewire body comprised of a first hypotube, a window attached to the first hypotube, and a flexible tip attached to the window. A second hypotube assembly forms an imaging core comprised of a second hypotube, one or more optical fibers arranged along an inner surface of the second hypotube, and a proximal connector attached to a proximal end of the second hypotube. The proximal connector is configured to connect the imaging core to a patient interface unit, wherein the second hypotube is nested inside the first hypotube, such that to acquire an image of a vessel region, the second hypotube is pulled back and pseudo rotated, or only pulled back, with respect to the first hypotube, while the one or more core optical fibers scan the vessel region with light of one or more wavelengths transmitted through the window.

### CROSS-REFERENCE TO RELATED APPLICATIONS

n/a

### BACKGROUND INFORMATION

### Field of Disclosure

The present disclosure generally relates to medical devices. More particularly, the disclosure is directed to novel imaging guidewires configured for use in minimally invasive surgical (MIS) procedures, particularly coronary angioplasty.

### Description of Related Art

Catheters comprise thin elongated tube-like (tubular) instruments made of biocompatible materials and configured to be inserted into a patient's body for diagnostic or therapeutic purposes in MIS procedures known as “catheterizations”. A variety of medical catheterization procedures involve the use of a guidewire over which a flexible catheter can be threaded and guided, so the catheter can reach an intended target location inside the patient's body. A guidewire is a very thin wire typically made of stainless steel, nitinol (titanium-nickel alloy), or other similar biocompatible materials designed to navigate through the patient's anatomy (e.g., from an access artery to a vessel) and quickly reach a lesion area (vessel segment) under image-guided control. Once the tip of the guidewire has been navigated to the desired location inside the patient's body, the catheter can be threaded over and along the guidewire, with the guidewire providing support and guidance for the flexible catheter.

Metallic guidewires are generally classified as “workhorse wires” and “CTO wires”. Workhorse wires are designed to provide support, torque, and lubricity for passage through various complex anatomies, as well as to allow delivery of stents, catheters, balloons, and other devices. CTO wires are designed to cross complex lesions often associated with a medical condition called Chronic Total Occlusion (CTO), which is a complete or nearly complete blockage of one or more coronary arteries. Crossing CTO lesions requires a set of dedicated CTO guidewires as well as specialized technical skills. Therefore, it is not uncommon to exchange one set of guidewires for another (e.g., exchange a workhorse wire for a CTO wire) during a complex procedure, which is done usually through a guide catheter. A guide catheter is pre-shaped catheter configured to quickly access the ostium or opening to both major coronary arteries.

There are various types of endovascular catheterization procedures, which typically include a large number of steps including various types of imaging. Fluoroscopic images and electronic means such as Electrocardiograms (EKG) initially guide clinicians to obvious narrowing of the coronary arteries, which can then be diagnosed and treated in more detailed Percutaneous Coronary Intervention (PCI), basically catheterization thru the skin.

Intravascular catheterization includes inserting a guidewire through an access artery (e.g., brachial, femoral, or radial artery), passing the guidewire to a vessel of interest within the vasculature, and then sending catheters over the guidewire. A cerebral catheterization procedure, for example, involves inserting a catheter into an artery in the leg of a patient, and passing the catheter over a guidewire up to the blood vessels in the brain. The use of a guidewire is intended to speed up the procedure, and, more importantly, reduce the risk of trauma to the patient caused by the tip of the advancing catheter. A guidewire can also act as a rail over which other intravascular devices such as an angioplasty balloon or a stent can be delivered to a site of interest. Therefore, when the initial catheter must be exchanged for other intravascular device or for another catheter of a different size or type, the first catheter has to be withdrawn over the guidewire, and a second catheter or other intravascular device is slid into place over the same guidewire. Depending on the procedure, additional catheters or additional intravascular devices may be repeatedly guided over the same guidewire. This process can lead to lengthy procedural times which may be detrimental to a patient's health. In order to ameliorate catheter exchangeability and reduce the length of procedural time, improved catheter and guidewire designs are desired.

One such improvement has been the development of guidewires with imaging capabilities. Ultrasound imaging guidewires are known from, for example, U.S. Pat. Nos. 5,095,911 (to Pomeranz), 5,368,035 (to Hamm, et al.), 6,078,831 (to Belef et al.), 6,529,760 (to Pantages, et al.), among others. Optical imaging guidewires are known from, for example, U.S. Pat. No. 6,134,003 (to Tearney, et al.), and pre-grant patent application publication US 2020/0183083 (by Tasker, et al), among others. The disclosures of these and other patent publications listed in the present disclosure are incorporated by reference herein in their entirety.

In general, one of the main purposes of a guidewire (either imaging or non-imaging guidewire) is to gain access to blood vessels using a minimally invasive technique. Therefore, a guidewire should have stable torqueability, pushability, and excellent kink resistance to achieve good control of the guidewire body for efficient catheter navigation. To that end, the guidewire body should also be laterally stiff to provide proper guidance for catheters, which are generally much more stiff (laterally rigid) than guidewires. A conventional imaging guidewire uses a rotatable imaging core operated by a drive cable, which receives torque from a torque source, such as a motor located outside of the guidewire.

In conventional imaging guidewires that use a rotatable imaging core, sometimes the system generates too much information that can overwhelm the user with excessive data that the user does not necessary need to see in preliminary stages when the guidewire is inserted into a lumen. For example, in a conventional intravascular OCT imaging guidewire, the probe rotates with a frequency of 100 revolutions per second (i.e., 100 Hz). If a single pullback is performed with a speed of 20 mm/s and lasts 2.7 s, the pullback allows for imaging of up to 72 mm of the vessel during a single pullback of the probe. Such a fast pullback of the OCT probe produces significantly large amounts of information (e.g., more than 50,000 axial lines of OCT signal), which the clinician does not really need to see when assessing the location and size of the stenosis to determine the appropriate stent. Such excessive information in an image of an anatomical vessel is likely unnecessary, but the clinician still has to record, review, and report on everything he/she sees. This makes the clinician's job harder than is necessary.

In addition, a rotatable imaging core of an imaging guidewire can experience non-uniform angular velocity that can cause Non-Uniform Rotational Distortion (NURD) in the images. Specifically, a rotatable imaging core of an imaging guidewire can experience lack of angular fidelity (a mismatch between rotation of the proximal end and the distal end) that can distort the image, and mislead the clinician in the interpretation of imaging results. Therefore, in addition to the overall properties of a non-imaging guidewire, the guidewire body of an imaging guidewire should have sufficient torsional resistance to minimize NURD. Furthermore, when multiple catheters have to be swapped during a procedure, attaching and detaching the imaging core to/from its torque source device can cause the guidewire to get soiled during catheter swap(s). Therefore, there remains a need for improved imaging guidewires.

### SUMMARY OF EXEMPLARY EMBODIMENTS

An objective of the present disclosure is to streamline percutaneous transluminal coronary angioplasty (PTCA) also called percutaneous coronary intervention (PCI) procedures, by eliminating at least some of the current steps that become unnecessary due to improved capabilities of a novel imaging guidewire disclosed herein. Another objective of the present disclosure is to reduce the amount of data acquired and displayed, while still obtaining clinically useful images that accurately convey the state of an area of interest such as a stenosis, in all stages; when the guidewire is inserted into a lumen to assess the location and size of the stenosis to determine the appropriate procedure, and also to assess the proper sizing and deployment of a stent.

According to at least one embodiment, the present disclosure describes an imaging guidewire that has mechanical properties comparable to a standard workhorse guidewire or a CTO guidewire, and uses radiative energy to assess vessel lumen size and vessel morphology, without the need for multiple device swaps. Thus, the present disclosure provides a novel imaging guidewire configured to function well as a guidewire platform for therapy delivery, and as an imaging modality for assessing vessel stenoses and morphology.

In one embodiment, an imaging guidewire is inserted into a biological lumen and configured for over-the-wire catheter exchanges without removal of the guidewire from the biological lumen. The imaging guidewire comprises: two hypotube-based assemblies including a first hypotube assembly and a second hypotube assembly, which is nested and telescopically translatable with respect to the first hypotube assembly. The first hypotube assembly includes a first hypotube and a cylindrical window that form a guidewire body. The second hypotube assembly includes a second hypotube and one or more optical fibers that form an imaging core. A tubular shaft having a proximal end and a distal end with a lumen extending therethrough along a central axis thereof, the tubular shaft comprising a first hypotube and a window section, the window section being made of a material substantially transparent to radiative energy suitable for propagation through tissue of a subject; an imaging core composed of a second hypotube and one or more optical fibers configured to acquire an image of a biological lumen through the window section; a proximal connector arranged at the proximal end of the tubular shaft and configured to connect the imaging core to a patient interface unit (PIU), wherein the second hypotube assembly is nested inside the first hypotube assembly such that, to acquire an image of the biological lumen, the imaging core is telescopically movable with respect to the tubular outer shaft of the imaging guidewire.

An imaging guidewire configured to guide a catheter into a bodily lumen of a subject, and to acquire an image of a target region of the bodily lumen, the imaging guidewire comprising: a guidewire body having, in order from a proximal end to a distal end, a rigid hypotube section, a semi-rigid hypotube section, and a window section with a large lumen extending from the proximal end to the distal end of the window, and a floppy tip attached to the distal end; a core hypotube having a proximal end and a distal end and arranged in the central lumen, the core hypotube having one or more optical fibers arranged lengthwise in the lumen of the core hypotube such that a distal end of each of the one or more optical fibers protrudes distally from the distal end of the core hypotube and is aligned with the window section of the guidewire body; a proximal connector attached to the proximal end of the core hypotube and configured to hold a proximal end of each of the one or more optical fibers at a first plane on the proximal end of the proximal connector, wherein the proximal connector is further configured to connect the core hypotube to a patient interface unit (PIU) such that each of the one or more optical fibers can receive radiative energy suitable for irradiating the target region of the bodily lumen, and wherein the core hypotube is nested inside the guidewire body such that, to acquire an image of the target region of the bodily lumen, the core hypotube is rotated and/or translated with respect to the guidewire body to cause each one of the one or more core optical fibers to scan the target region with a beam of the radiative energy that is transmitted through the window section.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Note that all illustrations are foreshortened for convenience and ease of illustration. The following drawings are illustrative of particular embodiments and are presented to assist in providing a proper understanding of those skilled in the art of medical devices. The drawings are not to scale (unless so stated) and are intended for use in conjunction with the explanations in the following detailed description. The present invention will hereinafter be described in conjunction with the appended drawings, wherein like numerals denote like elements and functions.

FIG. 1 illustrates a medical system 700 configured to acquire intraluminal data from a bodily lumen (e.g., a vessel 740) using an imaging guidewire 100;

FIG. 2 illustrates a schematic view of the imaging guidewire 100 according to an embodiment of the present disclosure;

FIG. 3 illustrates details about the manner in which a proximal connector 111 is attached to an imaging core 200, and details telescopic movement of the imaging core 200 with respect to the guidewire body 120;

FIG. 4A and FIG. 4B illustrate a non-rotatable imaging core 200 comprised of a plurality of core optical fibers 202;

FIG. 5A and FIG. 5B show cross-sectional views of a stiffness transition zone where a slotted hypotube section 130 (on the left) transitions into the window section 140 (on the right);

FIG. 6 illustrates details about the distal tip 160 (a floppy tip and an atraumatic cap) arranged distally to the window section 140, at the distal end of the guidewire body;

FIG. 7A is a side-view of imaging guidewire 100 along a y-z plane, showing a proximal connector 111 covered by a protective sleeve 113. FIG. 7B is a top-view of imaging guidewire 100 seen along an x-z plane, showing the protective sleeve 113 removed from the proximal end of the guidewire body. FIG. 7C is a side-view of the non-rotatable imaging core 200, showing multiple radiation beams configured to scan a vessel 740;

FIG. 8A is an isometric view, and FIG. 8B is a plan view of the proximal connector 111 which shows the arrangement of a plurality of core optical fibers 202 at the proximal end of the imaging core 200;

FIG. 9A, FIG. 9B, and FIG. 9C are schematic representations of distal optics assembly 209 used on core optical fibers, according to the various embodiments disclosed herein;

FIG. 10 shows a distal view of the imaging core 200 where a plurality of core optical fibers 202 scan a vessel 740 with a plurality of light beams to acquire image data of the vessel;

FIG. 11 illustrates an embodiment of a PIU 720 configured to control the imaging guidewire 100 having a non-rotatable imaging core 100;

FIG. 12 illustrates an exemplary wireframe image 1200 reconstructed from vessel data collected by system 700 controlling the non-rotatable imaging core 200 of imaging guidewire 100, and displayed in a longitudinal or L-mode format;

FIG. 13A illustrates an embodiment of a imaging guidewire 100 including a imaging core 300 operated by a dual-drive rotation-by-translation system;

FIG. 13B shows an embodiment of the imaging core 300 having a proximal connector 111, and configured to scan a vessel 740 using a single beam of radiative energy irradiated in a helicoidally moving path;

FIG. 14 illustrates an embodiment of a PIU 720 configured to control the imaging guidewire 100 having an imaging core 300;

FIG. 15 illustrates an exemplary image 1500 reconstructed from vessel data collected by the system 700 controlling the imaging core 300 of imaging guidewire 100, and displayed in a longitudinal or L-mode format;

FIG. 16 illustrates an embodiment of a PIU 720 configured to control the imaging guidewire 100 having an imaging core 500; and

FIG. 17 illustrates an exemplary image 1700 reconstructed from vessel data collected by the system 700 controlling the imaging core 500 of imaging guidewire 100, and displayed in a 3D isometric view.

### DETAILED DESCRIPTION OF EXEMPLARY EMBODIMENTS

The following description of examples and embodiments of an imaging guidewire should not be used to limit the scope of the claims. Throughout the figures, the same reference numerals and characters, unless otherwise stated, are used to denote like features, elements, components or portions of the illustrated embodiments. In addition, while the subject disclosure is described in detail with reference to the enclosed figures, it is understood that changes and modifications can be made without departing from the true scope of the disclosure as defined by the appended claims. Although the drawings represent some possible configurations and approaches for enabling the claimed subject matter, the drawings are not necessarily to scale and certain features may be exaggerated, removed, or partially sectioned to better illustrate and explain certain aspects of the present disclosure. The description set forth herein is not intended to be exhaustive or otherwise limit or restrict the claims to the precise forms and configurations shown in the drawings.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise (e.g. 1 to 5 includes 1, 1.5, 2, 2.75, 3, 3.80, 4, 5, and any values therebetween). As used herein, the term “substantially” is meant to allow for deviations from the descriptor that do not negatively affect the intended purpose or function. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the actions and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams may be presented in a sequence(s), it should be understood that the various operations may be performed in other order than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

The present disclosure generally relates to medical devices, and it exemplifies embodiments of an imaging guidewire. The imaging guidewire includes an elongated flexible shaft including an optical imaging core which may be applicable to a spectroscopic apparatus (e.g., an endoscope), an optical coherence tomographic (OCT) catheter, or a combination of such elongated apparatuses (e.g., a multi-modality imaging catheter). The embodiments of elongated apparatuses and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

In the present disclosure, the terms “optical fiber”, “fiber optic”, or simply “fiber” refers to an elongated, flexible, light guiding component or optical waveguide capable of conducting light from one end to another due to the effect known as total internal reflection. The terms “light guiding component” or “waveguide” refer to, or may have the functionality of, an optical fiber. The term “fiber” may refer to one or more light conducting fibers. An optical fiber has a generally transparent, homogenous core, through which the light is guided, and the core is surrounded by a homogenous cladding or multiple claddings. The refractive index of the core is larger than the refractive index of the cladding. Depending on design choice some fibers can have multiple claddings surrounding the core. In an imaging guidewire, optical fibers can be used to acquire images of bodily lumens (e.g., a blood vessel) based on scattering properties and/or fluorescence of bodily tissue (e.g., vessel tissue). For example, an imaging guidewire can be used in a multi-modality OCT-fluorescence system (MMOCT system). In an OCT-fluorescence system, a double-clad fiber (DCF) is used to deliver illumination light from an OCT light source and fluorescence excitation light from an excitation source while collecting the backscattered OCT signal through the single-mode core and fluorescence emission through the inner cladding of the DCF. In some embodiments of an OCT-fluorescence system, a multi-clad fiber can be used. The first layer of cladding may collect backscattered light, while the additional cladding can serve as an additional ‘channel’ for collecting certain information such as color or tissue fluorescence.

In the present disclosure, the term “imaging guidewire” refers to a guidewire that functions as a conventional guidewire (e.g., a workhorse wire or CTO wire), but also functions to acquire image data from a biological lumen such as a vessel. The term “biological lumen” or “bodily lumen” refers to the inside space of a tubular structure or a body of a patient, such as an artery or intestine. The term “patient” is generally synonymous with the term “subject” and includes all mammals including humans. Examples of patients include humans, livestock, and companion animals. The term “vasculature” refers to blood vessels, such as veins and arteries, in the body or in an organ or body part of a patient. Although portions of this disclosure may refer to veins and arteries, this disclosure is applicable to any type of blood vessel within the “vascular system.”

The term “hypotube” refers to a long metal tube (hypodermic tube) with or without micro-machined features along its length. In the present disclosure, the term hypotube is used to describe different components of the guidewire device. An inner or core hypotube is used to hold one or more optical fibers of the imaging core. An outer hypotube includes a solid-walled hypotube portion near the proximal end of the guidewire body, and a micro-machined helically-slotted portion of the same hypotube in the mid-section connected to a thin-walled. The solid-walled hypotube portion is used to enable imaging core pullback and re-advancement in open space within the catheter handle and PIU. The helically-slotted portion of the same hypotube is used in the mid-section to distal end to provide varying lateral stiffness along the length of the guidewire device.

In the present disclosure, by “translation” is meant a displacement of an object from one position to another in such a manner that all points of the object traverse equal parallel paths. A motion of translation is generally called linear motion. By “rotation” is meant a displacement of an object where all points of the object trace circular paths about a single axis often known as the axis of rotation. The axis of rotation of an object may pass through the object or may be outside of the object. A motion of rotation is generally called angular motion. A “torque” is a force capable of producing a change in angular velocity of an object. The numerical measure of the turning effect of a force that produces a change in angular velocity of an object is the “moment” of the force. The term “pseudo rotation” is used to describe displacement of an object about an axis of rotation as a result of a force other than a direct torque force. For example, in one embodiment, pseudo rotation is described as rotation-by-translation where linear translation of an inner hypotube with respect to an outer hypotube causes the inner hypotube to rotate a limited number of revolutions inside the outer hypotube.

### <System Overview>

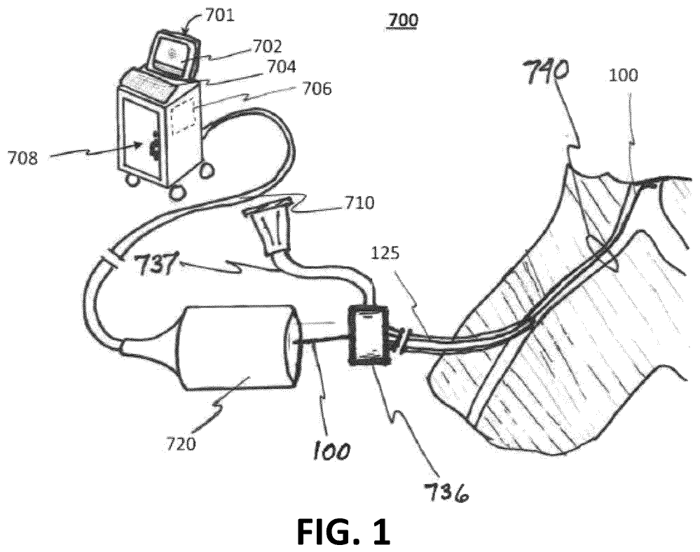

FIG. 1 illustrates a medical system 700 configured to acquire intraluminal data from a biological lumen (e.g., a blood vessel) using an imaging guidewire 100. The medical system 700 (herein after “system 700”) includes a system console 701, a patient interface unit (PIU) 720, and an imaging guidewire 100. The imaging guidewire 100 is a disposable (non-reusable) medical device connectable to the PIU 720 via a proximal connector 111 (not shown in FIG. 1). A custom guide catheter 125 designed to access coronary arteries is inserted into the vessel 750, and the distal tip of the catheter is seated in the ostium, the opening to the right or left coronary artery. Then, the guidewire 100 is inserted and advanced into the vessel of interest until the imaging plane is distal to the stenosis of interest. The system console 701 is connected to the PIU 720 via an umbilical 710. The umbilical 710 includes a cable bundle comprised of electrical wires, optical fibers, and other components (e.g., tubing for fluids), etc. An offline workstation for image analysis and storage may also be available. The PIU is the interface between the imaging guidewire 100 and the console 701. The PIU provides the means to pullback the imaging core within the guidewire body that remains stationary. During use, the PIU 720 is preferably covered with a sterile drape and placed on the patient bed.

The console 701 may include, among other components, a computer 706, a display 702 (e.g., LCD or OLED screen), an input device (e.g., keyboard, touchscreen, mouse), and other hardware components 708. The console 701 is configured to control the overall functions of the imaging guidewire 100, and to provide a user interface (e.g., via display 702 and keyboard 704) for interaction of the user (a physician) with the system 700. The computer 706 can include one or more processors, such as a central processing unit (CPU) and well known peripheral components (not shown). Computer 706 refers to any machine that operates to accept a structured input, processes the structured input according to prescribed rules, and produces one or more results as output. Hardware components 708 may include system components such as a source of radiative energy (one or more light sources), an interferometer, one or more detectors, optical systems, and other electronic peripherals.

In an OCT system for intravascular procedures, the source of radiative energy can include a short coherence length or frequency tunable light source. For example, a laser source (specifically, a swept source laser) can be used to emit ultrafast pulses of near-infrared light that can be used to acquire OCT images of a patient's vessel. Hardware components 708 may also include parts of the OCT interferometer, e.g., optical components of the reference arm, delay lines, etc. OCT images can be acquired by scanning a vessel 740 with an imaging core arranged inside the imaging guidewire 100. As used herein the term “scan” or “scanning” refers to the process, act, or instance of passing a beam of electromagnetic radiation over or through an object to systematically send and receive radiation in order to obtain data indicative of morphology and/or chemical composition of the object. To that end, the PIU 720 is configured to removably connect the imaging guidewire 100 to the system console 701. The PIU 720 includes optical, mechanical, and electronic components which are not shown in FIG. 1A but are well known to persons of ordinary skill in the art of conventional OCT systems. The PIU 720 includes a first gripping unit 727 (e.g., a female connector) configured to receive and mechanically grip the outer shaft (guidewire body) of imaging guidewire 100, when the guidewire is inserted into the PIU 720.

In an intravascular procedure, the imaging guidewire 100 is inserted into a blood vessel 740 via an introducer and/or guide catheter 125, as shown in FIG. 1A. Then, the distal end of imaging guidewire 100 is guided to a site of interest (e.g., a stenosis) through the guide catheter 125, which is attached to a hemostasis valve 736. A side arm with a luer taper 737 allows the injection of liquids such as drugs or contrast agent thru the guide catheter 125. An additional imaging modality such as an X-ray fluoroscopy system or a computed tomography (CT) system can be used to monitor the insertion and positioning of the imaging guidewire 100 into the blood vessel 740. When the distal end of the imaging guidewire 100 is placed beyond the stenosis at the site of interest, the imaging core undergoes a pullback operation whereby the imaging core scans the vessel 740 through a transparent window section of the guidewire body to acquire images of the vessel wall without moving the guidewire in the vessel.

Conventional imaging guidewires and catheters typically use a rotating imaging core (e.g., a rotatable optical fiber with a distal optics assembly arranged near the distal end of the catheter or guidewire body) operated by a motor to scan a biological lumen (e.g., a coronary artery) with electromagnetic radiation in a known manner. Most guidewires also include a distal tip, sometimes referred to as a “floppy tip”, which is a soft and flexible atraumatic segment attached to the distal end of the guidewire body. The floppy tip can be pre-shaped, or can be manually bent by the user to enhance navigational capabilities and prevent injury to the patient.

In accordance with the various embodiments disclosed herein, the imaging guidewire 100 is significantly different from conventional imaging guidewires in that the present guidewire is designed to eliminate and/or significantly reduce NURD. The imaging guidewire 100 is constructed with nested hypotubes that telescopically move with respect to each other to effect imaging without moving or rotating the body of the guidewire within the biological lumen. The guidewire is particularly designed to generate a reduced amount of image data which alleviates the operator's burden and reduces the duration of a procedure. In an OCT-NIRAF modality, the imaging guidewire includes a guidewire body with an imaging core. The imaging core includes one or more than one double-clad fiber (DCF) to deliver OCT source light and fluorescence excitation light to a vessel while collecting backscattered OCT light and fluorescence emission through the same imaging core to generate co-registered OCT and fluorescence images. Notably, the light is collected by distal optics of each fiber, and OCT light is channeled through the single mode core, whereas fluorescence emission is channeled through the inner cladding of the DCF.

In one embodiment, the imaging guidewire 100 includes a guidewire body 120 with a flexible tip 160, and a non-rotatable imaging core 200 arranged inside the guidewire body. The non-rotatable imaging core 200 includes a plurality of optical fibers that can scan a vessel wall without core rotation. The use of a non-rotatable imaging core eliminates the need for a rotatable drive cable which also eliminates non-uniform angular velocity that can cause NURD in the images. This embodiment creates a basic wire-frame image with sufficient data to assess the location and size of the stenosis to determine the appropriate stent, without overwhelming the user with excessive data that the user does not necessarily need or want to see. The imaging guidewire 100 having a non-rotatable imaging core 200 and including multiple optical fibers that can scan a vessel wall without core rotation can generate a basic wire-frame image without NURD. This provides an effective solution to one or more shortcomings of conventional imaging guidewires.

In other embodiment, the imaging guide wire 100 includes a guidewire body 100 with a pseudo rotatable imaging core 300 driven from both the proximal and distal ends through a limited number of rotations. In this case, the PIU drives the proximal end of the imaging core at precisely the pitch of the dual-drive, a threaded element near the distal end, in effect driving the core from both ends. The pseudo rotatable imaging core 300 includes at least one double-clad fiber rotated by a dual-drive rotation-by-translation system, which appropriately reduces non-uniform rotation artifacts and/or controls angular velocity to avoid NURD in the images. In a further embodiment, the imaging guidewire 100 includes a pseudo rotatable imaging core 500. The pseudo rotatable imaging core 500 includes functions of the rotation-by-translation dual-drive system and uses a plurality of double-clad fibers (DCF) to scan a vessel wall. The imaging core 500 delivers OCT source light and fluorescence excitation light to a plurality of locations of a vessel wall while collecting backscattered OCT light through the single-mode core and fluorescence emission through the inner cladding of each of the DCFs. In all embodiments, the guidewire body 120 includes, in order from the proximal end to the distal end, a rigid hypotube section, a semi-rigid hypotube section, and a semi-flexible window section with a hyper-flexible floppy tip attached to the window section. The semi-rigid hypotube section is a laser-cut slotted hypotube section that offers a smooth stiffness transition zone between the semi-rigid hypotube section and the semi-flexible window section, which is advantageous for navigating through tortuous vessels. In that regard, the guidewire disclosed herein can be used during minimally invasive diagnostic and therapeutic procedures in the cerebrovascular, cardiovascular, and peripheral vascular systems.

In contrast to a conventional OCT system, a dual-mode imaging system adds the capability to detect fluorescence to the OCT system by focusing the appropriate wavelength excitation light at the vessel being imaged, and channeling the emitted light thru the first cladding layer (clad) back to the system for analysis and eventual display to the clinician. Each type of plaque on vessel walls emit a unique signature wavelength when excited with certain wavelength light. Additionally, in addition to the hardware and coupling variations, the PIU has the capability to grip the imaging guidewire in at least two locations, and efficiently couple excitation light into guidewire fibers sequentially. The imaging guidewire system includes equipment and software capability to display the image from a non-rotating, or limited-rotation (dual drive) core imaging pullback. This includes an optical encoder 732 aligned with the connecting fiber 726 in the PIU, which is rotating during imaging to sequentially excite each core optical fiber 202 one at a time during the pullback. The encoder 732 tells the system which fiber is being excited so the system knows where to display the resulting data forming an image of the vessel lumen, including tissue fluorescence information useful to the clinician. An example of tissue information not available via a conventional OCT imaging system includes plaque types and other vessel wall features such as the location and size of lipid pools and their fibrous caps. These hard to see lesions can rupture, causing a local blood clot or thrombus that can cause adverse events on a patient. It is known that placing a stent end over a lipid pool can cause such adverse events during a catheterization procedure. This is one of the primary justifications for the use of fluorescence-detecting imaging in the heart. An exemplary application of system 700 is for acquiring optical coherence tomography (OCT) images of coronary vasculature for diagnosis and/or treatment of patients with coronary diseases and related conditions. Intravascular OCT is a high-resolution interferometric imaging modality that uses backscattered or reflected light to produce cross-sectional images of a blood vessel with micrometer resolution. The system 700 can also be applicable to other catheter-based imaging modalities, such as intravascular ultrasound (IVUS) imaging, near-infrared auto-fluorescence (NIRAF) imaging, near-infrared spectroscopy (NIRS) imaging, and multimodality imaging techniques such as an OCT-NIRAF catheter system, a NIRS-IVUS catheter system, and other combinations thereof. In an OCT-NIRAF system that produces an image of a vessel, light of short coherent length is delivered from an OCT light source and light suitable for exciting fluorescence is delivered from an excitation light source a blood vessel through an imaging core of the imaging guidewire. A fiber optic rotary joint (FORJ) is used to transfer light from the system light source to a rotating connector, so that the imaging core can scan the vessel wall with multiple beams beam of light delivered by multiple fibers in the imaging core. Light reflected, scattered, and or emitted by the vessel is collected by the imaging core, and sent to a processing device, such as computer. The collected signal is processed by a computer to produce cross-sectional or 3D wire-frame images of the biological lumen. In addition, backscattered or emitted tissue fluorescence light can be collected and processed to acquire information about tissue morphology.

### <Imaging Guidewire>

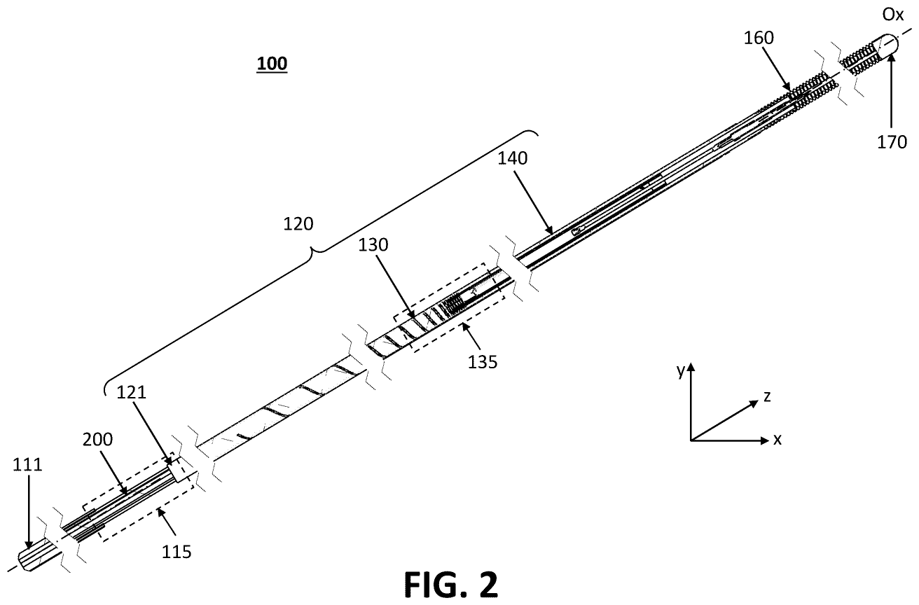

FIG. 2 through FIG. 6 illustrate various details of the imaging guidewire 100, according to one or more embodiments of the present disclosure. Imaging guidewire 100 is comprised of an outer tubular portion referred to as guidewire body 120 which is fitted with a window segment 140 and a floppy tip 160. Inside the guidewire body 120 is the imaging core 200, which is retractable with respect to guidewire body 120. The guidewire body 120 is shown as an elongated tubular shaft spanning from a proximal end (left-hand side) to a distal end (right-hand side) with a central lumen that spans from a proximal end to distal end along a longitudinal axis (Ox). In a three-dimensional (3D) environment defined by Cartesian coordinates (x, y, z), the imaging guidewire 100 extends in a lengthwise direction along the z-axis, and the cross-section of the guidewire is on the x-y plane (perpendicular to the z-axis).

The imaging guidewire 100 includes a guidewire body 120 comprised of proximal shaft section, a mid-shaft transition section, a distal shaft section. Distally to the distal shaft section, a shapeable or pre-shaped distal tip 16o is fixedly attached to the guidewire body 120. The guidewire body 120 has a length, and the interior central lumen extends lengthwise through the proximal shaft section, the mid-shaft section, and reaches the distal shaft section, but not reach the distal tip. The distal shaft section includes a cylindrical window made of a material substantially transparent to imaging radiation. The interior central lumen (guidewire lumen) is configured to slidably receive the imaging core according to the various embodiments described herein. In one embodiment, the imaging core 200 is comprised of a core hypotube 201 and a plurality of core optical fibers 202 arranged inside the core hypotube. The imaging core 200 can translate linearly with respect to the guidewire body 120, and scan a vessel wall with substantially orthogonal beams of imaging radiation emitted from each of the core optical fibers 202 and transmitted through the window section. The surfaces of imaging guidewire 100 and imaging core 200 may be coated in its entirety with a lubricious coating material that enables improved movement of the guidewire body 120 within a patient's anatomy (e.g., a vessel). At the proximal end of the guidewire body 120, a proximal connector 111 is attached to the imaging core 200, and the proximal connector 111 is configured to connect the imaging core 200 to PIU 720.

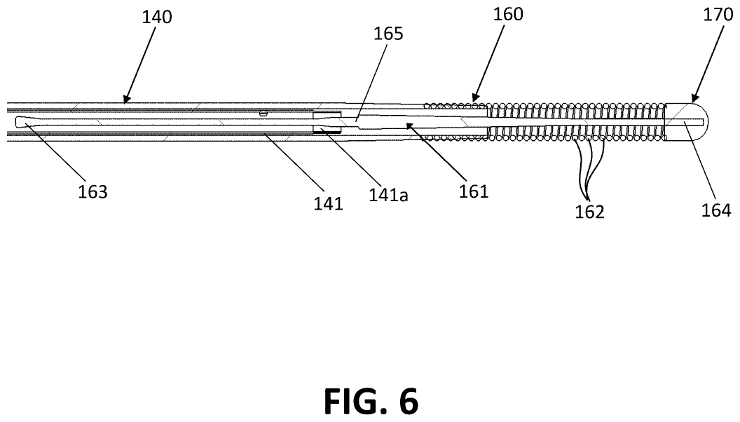

In FIG. 2, imaging guidewire 100 is shown with distal and proximal ends in cross-section, and the body of the guidewire is not in cross section. A first joint portion 115 shows the imaging core 200 pulled back, exposing the smaller core hypotube between larger diameter proximal connector and guidewire body 120; this illustrates the manner in which the proximal connector 111 is attached to imaging core 200. A second joint portion 135 shows the manner in which the slotted hypotube section 130 is joined with the window section 140. FIG. 6 shows the manner in which the distal tip 160 is attached to the window section 140. On the distal end of the window section 140, the distal tip 160 includes coiled wires 162 that are progressively embedded in the solid window material in a direction from the distal end towards the proximal end. A lead-in or atraumatic cap 170 is formed at the far end (most distal point) of distal tip 160. Details about the distal tip 160 are shown and described in reference to FIG. 6.

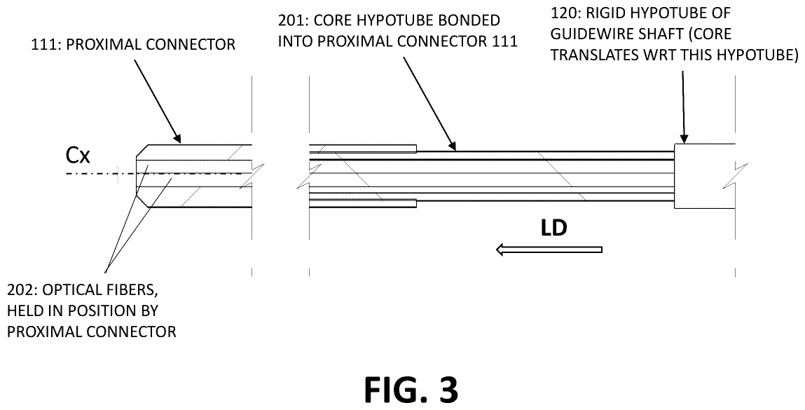

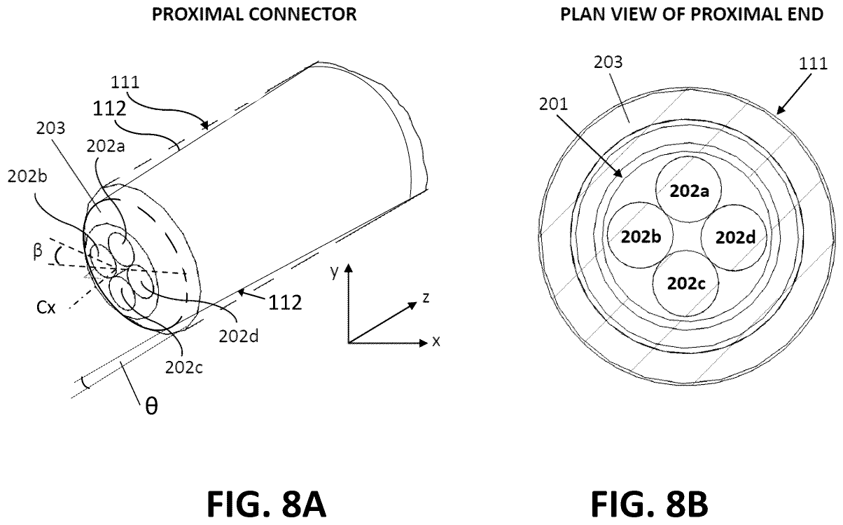

FIG. 3 shows a side view of the fiber-optic arrangement within the proximal connector 111. The proximal connector 111 is a ceramic ferrule configured with a single inner diameter formed to snugly fit a plurality of core optical fibers 202. The proximal connector 111 is attached to the core hypotube 201, for example, by adhesive bond, laser weld, pressure fit, etc. Since the core hypotube 201 is fixedly attached to the proximal connector 111, when the imaging core 200 is pulled back, the core hypotube 201 translates in a linear direction LD with respect to the guidewire body 120. FIG. 3 illustrates an example of the manner in which the proximal connector 111 is attached to the core hypotube 201 at the proximal end of imaging core 200, and the manner in which the proximal ends of core optical fibers 202 are held by the proximal connector. The proximal connector 111 holds the proximal ends of core optical fibers 202 arranged around its central axis Cx, which is preferably collinear with the longitudinal axis Ox of the guidewire body (see also FIG. 8A). Since the core hypotube 201 is nested inside the guidewire body 120, when pullback occurs, the core hypotube 201 moves linearly with respect to the guidewire body 120 (i.e., the core hypotube 201 moves telescopically with respect to the outer hypotube).

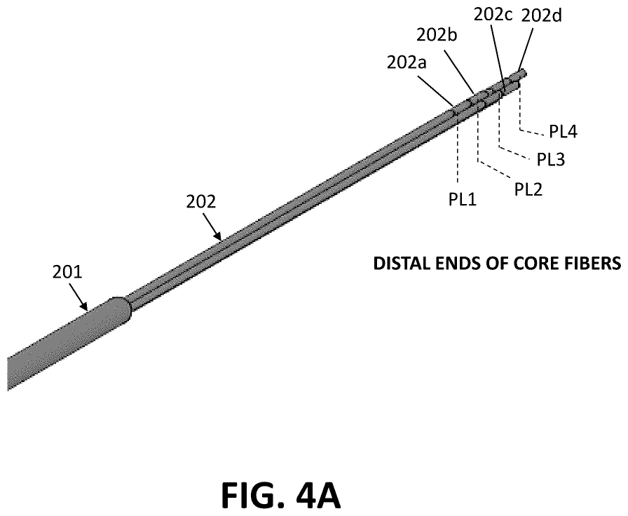

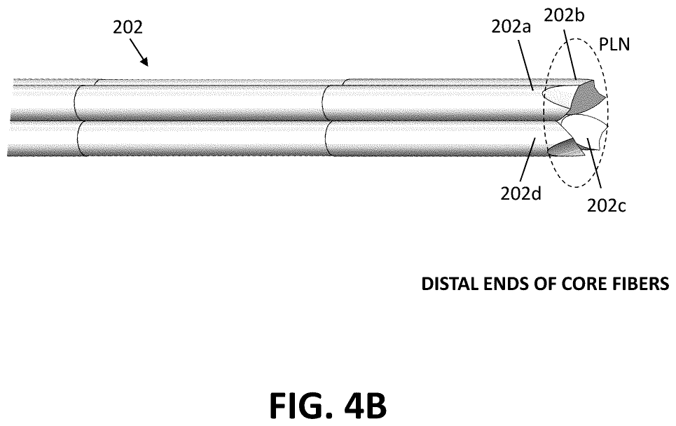

FIG. 4A and FIG. 4B shows additional details about the imaging core 200. In the example illustrated by FIG. 4A, the imaging core 200 includes a first core optical fiber 202a, a second core optical fiber 202b, a third core optical fiber 202c and a fourth core optical fiber 202d, which are at least partially enclosed by the core hypotube 201 and protruding through the distal end of the core hypotube 201, such that a distal end of each core optical fiber 202 is at a different plane (staggered in the lengthwise direction) with respect to the window section 140. On the other hand, as illustrated by FIG. 4B, the imaging core 200 includes the same number of core optical fibers 202, but the distal ends of all core optical fibers are arranged at a same plane.

Specifically, in FIG. 4A, at the distal end of core hypotube 201, the distal end of each core optical fiber 202 protrudes distally beyond the core hypotube 201 into the window section 140 where the distal ends of the fibers are staggered by a small distance such that a distal end of each fiber 202 is at a different plane in the lengthwise direction. For example, a distal end of first fiber 202a is located at a predetermined first plane PL1; a distal end of fiber 202b is located at a second plane PL2 distal to the first plane; a distal end of fiber 202c is located at a third plane PL3 distal to the second plane, a distal end of fiber 202d is located at a fourth plane PL4 distal to the third plane, etc. In contrast, in FIG. 4B, the distal ends of optical fibers 202a, 202b, 202c, and 202d are all arranged at a same distal plane PLN.

Although FIG. 4A and FIG. 4B show only 4 fibers 202 for illustration purposes, in some embodiments, there can be less (i.e., 3) or more (i.e., 8, 12, 24, 48, etc.) optical fibers arranged in either manner. In one exemplary embodiment, a plurality of 80-micron fibers (e.g., 48 core optical fibers of 80-micron diameter) are arranged inside the core hypotube 201. The core optical fibers 202 can be single mode, and/or multi-clad (e.g., double clad) optical fibers each with a distal optics assembly 209 at the distal end thereof.

In a configuration where the core fibers are staggered, to generate an image of the vessel 740, the system 700 can differentiate between signals collected by each one of the core optical fibers 202 via fiber length. In one embodiment, fiber lengths vary by a distance equal to or slightly greater than the diameter of the largest vessel in which the imaging guidewire 100 is expected to be used (for example the fiber lengths between the shortest and longest fiber may vary by about 1 cm or less). This difference in fiber lengths is enough for the system to be able to differentiate between fiber ends, and to know where a given fiber end is located with reference to the proximal connector (or with respect to the most distal fiber end), so that data collected from each fiber can be combined and merged into a single image with sufficient accuracy for clinical utility. That is to say, for example, the difference in fiber length should be such that an interference signal generated from each fiber can be combined into an image from which the clinician can gain enough valuable information to make an informed assessment on what diameter and length of a stent can be used to treat a stenosis.

The varying-length fibers configuration can include different number of fibers. In one embodiment, a small number of fibers, for example, 3 to 6 fibers (i.e., a minimum of three fibers) could be used to obtain a wireframe image. This is due to the fact that fiber length variation needs to be at least equal to or greater than the largest diameter vessel to be imaged, which is normally about 3-4 mm. So, in at least one embodiment, the difference in fiber lengths should be at least 5 mm between the shortest fiber and the longest fiber. If a larger number of fibers is employed, another fiber-differentiation method can be employed, such as discrete spectral bands that differ from fiber to fiber. This method would require spectral notch filters in the reference arm that match the spectral range for all fibers so the system can properly differentiate signals from the core fibers for image reconstruction. In other embodiments, multiple interferometers can be used, as discussed later.

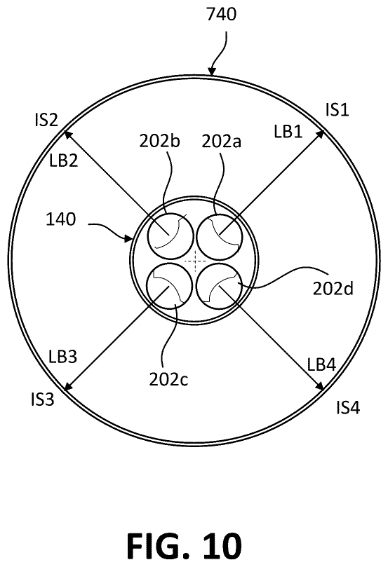

In a configuration where the core fibers are non-staggered (i.e., the fibers are at a same plane), to generate an image of the vessel 740, the system 700 can differentiate between signals collected by each one of the core optical fibers 202 by tracking which of the core fibers is excited, and collecting a signal from each fiber, as explained infra with respect to FIG. 10 through FIG. 12.

### <Transition Zone Between Slotted Hypotube and Window Section>

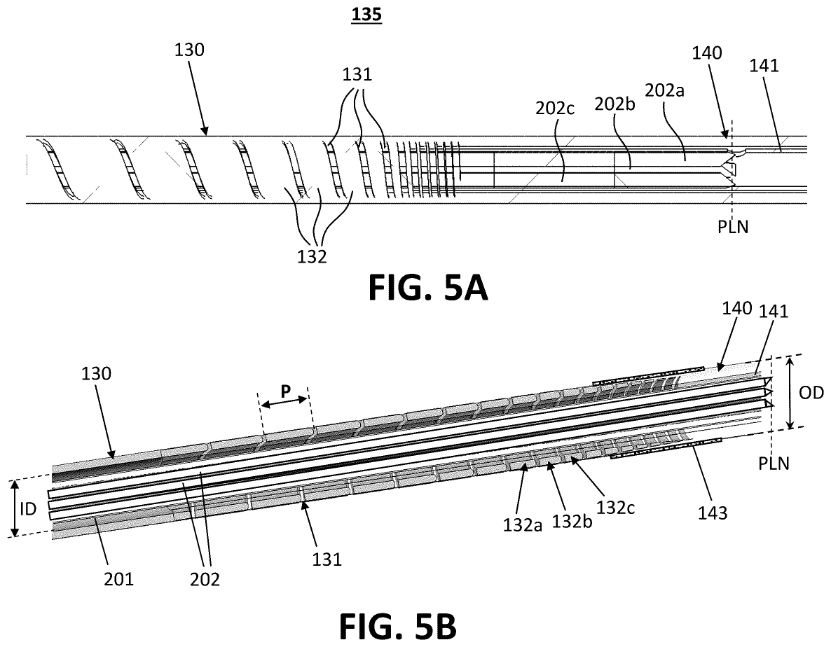

FIG. 5A and FIG. 5B show details of the imaging guidewire 100 at the stiffness transition zone (the second joint portion 135) where the slotted hypotube section 130 (on the left) transitions into the window section 140 (on the right). Here, the slotted hypotube section 130 seamlessly transitions into the window section 140 while maintaining an inner diameter (ID) and outer diameter (OD) substantially unchanged along the length of the joint portion. In order to optimize a wall thickness (WT=OD−ID) of the guidewire body, the inner diameter of core hypotube 201 is configured to maintain the imaging core 200 with a minimum tolerance inside the guidewire lumen. Further, in order to optimize torsional transition and lateral flexibility of the guidewire body, the slotted hypotube section 130 includes a predetermined pattern of laser-cut coils 132, where the coils 132 are arranged at a varying pitch. Lastly, to enable movement of the imaging core 200 with respect to the guidewire body 120, the core hypotube 201 is configured to move telescopically with respect to the outer hypotube forming the guidewire body 120.

Here, a varying pitch of a helical slot machined in the slotted hypotube creates different stiffnesses along the length of the slotted segment, with longer pitch equating to stiffer segments and shorter pitch creating softer stiffness towards the distal end of the guidewire body hypotube. Variations in pitch of the slotted hypotube helps create smooth stiffness transitions between various segments of the guidewire body that exhibit differing lateral stiffness. The guidewire body encloses the imaging core 200 comprised of a core hypotube 201 and one or more core optical fibers 202. The core hypotube is nested inside the solid-walled and slotted hypotube segments; and these nested hypotubes are telescopically movable with respect to each other. The terms “telescopically movable” or “telescoping” refers to the movement of one part sliding with reference to another part to change the overall length of an object (such as a telescope or the lift arm of a forklift platform). In conventional mechanical equipment, telescoping can be achieved by hydraulics or pulleys. In the present disclosure, telescoping of nested hypotubes can be achieved by manual movement (or by automated mechanical movement effected by the PIU) of one hypotube assembly with respect to another hypotube assembly.

According to at least one embodiment, e.g., as shown in FIG. 5B, the slotted hypotube section 130 can have a tapered wall which is laser-cut to form a pattern of narrow slits (slots 131) and a plurality of coils 132. More specifically, the slotted hypotube section 130 has a plurality of laser-cut coils 132 with slots 131 formed at a pitch (P). The pitch P is relatively large at the proximal end of the slotted hypotube section 130, and gradually changes to a smaller and smaller pitch towards the distal end of the slotted hypotube section 130. This variation in pitch P creates an increasingly softer lateral stiffness along the length of the guidewire body 120 in a direction from the proximal end to the distal end. In addition, the coils 132 are progressively etched to reduce the cross-section of the coils to facilitate sinking the coils into the window wall material to form a strong attachment between the hypotube and the window. The etching process includes a step of slowly dipping the laser-cut coils into etchant such that the more distal coils get etched the most, which reduces their cross-section the most. After etching, the coils are embedded into the wall of the window section, so that the most distal coils become fully encapsulated within the window wall. In the example shown in FIG. 5A, the laser-cut slot 131 has a predefined helicoidally oriented path that forms a plurality of helicoidal coils 132. FIG. 5B shows coils 132a, 132b, 132c have a substantially rectangular cross-section at a pitch P, where the pitch P decreases in a direction from the proximal end towards the distal end. In this embodiment, the pitch P varies in a direction of the proximal end to the distal end until the slotted hypotube section 130 is nearly as soft as the window section 140 to create a smooth stiffness transition between the two sections. One way to determine if the two sections have the desired stiffness is to measure the bending moment of each section. A bending moment (BM) is a measure of the bending effect induced in a structure when an external force (or moment) is applied to the structure, causing the structure to bend.

Lateral stiffness of the guidewire body varies significantly depending on the location along the length of the guidewire, and also between various types of guidewires. Some guidewires are soft, and others are super stiff to address certain clinical needs. In the present disclosure, at the distal tip 160, the floppy tip stiffness is general measured by how much force is needed to deflect the tip at 1 cm from the most distal end. To measure stiffness, weights of about 0.50 grams, or 0.50-0.90 grams (balanced), or 0.90-3.50 grams for an Amplatz-type wire intended for Chronic Total Occlusion (CTO) procedures can be used. The guidewire body can be configured to have a bending moment as low as 10 Mega-Pascal (MPa), but it can also be much stiffer for Amplatz-type guidewires used for CTOs, where guidewire body stiffness can be as high as 160 MPa. An MPa is a decimal multiple of a Pascal, which is the SI-derived unit of pressure, stress, Young's modulus, and ultimate tensile strength. A Pascal is a measure of force per unit area, defined as one newton per square meter. In this regard, guidewires are characterized by two factors: 1. Floppy Tip Stiffness, and 2. Device Support or stiffness of the guidewire body.

In one embodiment, the slotted hypotube section 130 is joined to the window section 140 by sinking the coils 132 into the proximal end of window section 140 with melt-bond tooling. Alternatively, for extremely thin hypotubes, the laser-cut pattern of the slotted hypotube section 130 can be etched into the window wall material and covered by a thin heat-shrinkable tube 143, simulating encapsulation, as shown in FIG. 5B. A thickness of the heat-shrinkable tube 143 needs only be sufficient to cover exposed coils 132 without substantially increasing the outer diameter (OD) of the guidewire body. This creates a strong mechanical attachment and smooth stiffness transition from the semi-rigid slotted hypotube section 130 to the softer polymer-based segment of the window section 140. Since the coils 132 of the slotted hypotube section 130 are sunk into the wall of the window section 140, the outer profile (OD) of the imaging guidewire 100 can be maintained substantially uniform and smooth.

In one embodiment, the slotted hypotube section 130 can have a continuous helicoidally laser-cut pattern, but the laser-cut pattern is not limited thereto. In other embodiments, as long as a strong mechanical attachment and smooth stiffness transition is achieved between the slotted hypotube section 130 and the window section 140, laser-cut patterns can be made of different shape or in different patterns. The laser-cut pattern of the slotted hypotube section 130 may vary depending on the medical procedure and the corresponding dimensions of the guidewire. For example, the laser-cut pattern of slotted hypotube section 130 may vary as a function of the size of the medical device, the location of the anatomy where the guidewire will be used, the length of the catheter required to reach a target location, etc. For example, instead of forming a continuous helicoidally oriented cut, the laser-cut slot 131 may include multiple discontinuous spiral slits following a helicoidally oriented path, as shown in FIG. 5A. In addition, in certain embodiments, the shape of the cut, the orientation of the cut, the size of the cut, etc., can be varied or alternated, so that a desired stiffness profile may be achieved in specific sections of the guidewire. Further in addition, the density (number) of cuts per unit length may be alternately increased and/or decreased to achieve the desired flexibility to the guidewire body.

Here, it is noted that the slotted portion of the slotted hypotube section 130 is relatively short when compared to the entire length of the guidewire body, but long enough to effectively create a smooth stiffness transition zone. In one embodiment, the length of the slotted portion could be as short as 2 centimeters (cm). In other embodiments, the length of the slotted portion could also be longer to achieve more flexibility with a smoother transition. Since the slotted portion of the hypotube usually remains inside a guide catheter, and as such is protected from tight bends, a longer slotted portion (e.g., 10-15 cm long) could be implemented in one or more embodiments of the present disclosure because the stiffness of the two sections differs so greatly, but smoother transition is achieved by progressively etching and maintaining the inner and outer diameter of the guidewire substantially constant.

The materials used to construct the guidewire body 120 may depend on the desired flexibility and pushability parameters of the guidewire. In one or more embodiments, the guidewire body 120 may be constructed of known metal materials such as, stainless steel, cobalt chrome, nitinol, or other similar metals or metallic compounds. In certain embodiments, the rigid hypotube section 121 and slotted hypotube section 130 may be formed of separate parts, where each part may be made of a different metal or a different metal alloy. Some known examples of metals and metal alloys suitable for flexible hypotubes include, but are not limited to, stainless steel, tungsten, nitinol; nickel-chromium alloy; nickel-chromium-iron alloy; cobalt alloy; tungsten alloys; copper beryllium, silver-plated-copper, and others. In addition, lubrication between nested hypotubes provides ease of telescopic movement during guidewire insertion and imaging core pullback. Therefore, lubricated, nested hypotubes, extrusions and hybrid hypo-extrusion combinations can be used to create smooth stiffness transitions between various segments of the guidewire body.

Varying the pitch P of the helicoidally oriented laser-cut slot 131 can provide various advantageous effects. For example, a longer pitch can provide more crush resistance due to longer segments of hypotube that resist crushing. On the other hand, a smaller pitch can provide more flexibility and ease of sinking the coils into the window section 140. The actual dimensions of the laser-cut slot 131 and the pitch parameters are not specifically defined here because these parameters can change according to specific applications, as it would be understood by those of ordinary skill in the art. In addition to varying the pitch of the coils, varying the kerf, or open slot between coils could also be increased toward the distal end of the slotted region to allow more space for the window material to melt into the gap between coils.

Materials for the guidewire body 120 (outer hypotube) can be chosen according to the type of guidewire application. For example, coronary imaging guidewires are generally provided as Intravascular Ultrasound (IVUS) imaging wires, or Optical Coherence Tomography (OCT) imaging wires. Both types of imaging guidewires can benefit from lubricious, low-friction materials because conventionally both utilize rotating imaging cores to acquire images of a patient's anatomy (e.g., coronary vessels). Images acquired by either type of imaging guidewire (IVUS or OCT) should ideally be free of image artifacts to facilitate accurate diagnosis of disease state(s). Although materials for fabricating slotted laser-cut hypotubes for IVUS and OCT imaging devices are well known, the difference in principles of operation between IVUS and OCT can significantly distinguish the material necessary to fabricate the window section 140 of an OCT device versus an IVUS device.

IVUS guidewires can acquire images through blood without the need for flushing or clearing the blood from the vessel. In an IVUS-type of imaging guidewire, the acoustic impedance (Z) of the window material should match the acoustic impedance of blood. Therefore, polyethylene (PE), particularly medium density PE is generally considered as a good example of an ideal imaging window material. If the acoustic impedance is not a good match for blood, there will be visible image artifacts that degrade the image and could mislead clinicians or make their job of evaluating images of Coronary Artery Disease (CAD) more difficult. This is due to inefficient transfer of acoustic energy through the window section 140, into blood and vessel tissue, and/or different types of plaque, etc., because the impedance is not well matched as acoustic energy propagates into vessel walls and echoes travel back through the window material and into the transducer.

Imaging guidewires for OCT imaging utilize electromagnetic radiation in a wavelength range of about 600 to 2000 nanometers (nm). Therefore, the refractive index (n) of the imaging window material is an important parameter to consider for fabricating imaging guidewires that can obtain artifact-free OCT images. In OCT imaging, because the average size of blood cells is close to the wavelengths of radiation commonly used in OCT imaging, and therefore prevents efficient light transmittance, blood is usually displaced by injecting flushing media for about one or two seconds for imaging. One example of flushing media used to displace blood is contrast agent. A type of contrast agent called Renografin, which is diatrizoate meglumine and diatrizoate sodium injection, is sometimes mixed 50/50 with saline solution, and used to displace blood during imaging. Therefore, the material for the window section 140 of an OCT imaging guidewire must be chosen by considering the wavelengths and refractive index of contrast agents most generally used in OCT imaging. Otherwise, similar to IVUS catheters, if the refractive index of the optical imaging window material is not well matched to that of the flushing media and/or tissue, imaging artifacts can interfere with optimal OCT imaging and diagnosis.

OCT imaging window materials should closely match the refractive index of contrast agents, blood, and/or vessel tissues. Transparent conductive polymers allow the passage of OCT imaging radiation thru the window section without excessive scattering or attenuation. In embodiments where transparent materials are used for the window section, longitudinal TiO2-doped stripes on or in the window material can be used for OCT calibration and also to detect and correct for imaging artifacts associated with rotating drive cables. An example of longitudinal strips provided on the window material is described in U.S. patent Ser. No. 10/952,702, which is incorporated by reference herein in its entirety. Commonly used window materials include certain grades of cyclic co-polymers (COPs) such as TOPAS® COC, certain grades of polyethylene, polypropylene, styrene, urethane, polycarbonate, PEBAX® block copolymers, PMMA, ULTEM, OKP-4 and Zeonex®, plus other semi-flexible materials commonly used for plastic optics. In certain embodiments, transparent aluminum (ALON™) can be utilized as a window material in at least part of the window section 140. In addition, transparent aluminum can be used to form rotational markers or stripes detectable with OCT for, e.g., catheter calibration.

An imaging guidewire 100 with a longitudinally translating imaging core, leaves an unsupported length of the window section 140 as the imaging core 200 is retracted for imaging pullback. Therefore, it is important that most of the lateral stiffness in the imaging region should come from the window structure, so that as the imaging core is pulled back, the guidewire body still retains most of its stiffness. In at least one embodiment of the imaging guidewire 100, a window support element—an inner support tube 141 that extends beyond the imaging plane—is used to help maintain stiffness of the guidewire body as the imaging core is retracted. In some embodiments where an ultra-stiff imaging window material such as ALON® transparent aluminum is used, the use of a supporting element (inner support tube 141) can be obviated.

Any material used for the window section 140 should have a refractive index between about 1.40-1.60, or preferably between about 1.47-1.57, and more preferably between about 1.50-157 which can help create artifact-free images due to their refractive index matching the refractive index of blood, contrast agents, and/or bodily tissues. In other words, according to at least one embodiment, the window section 140 can be made of transparent aluminum or polymeric material that substantially matches the refractive index of one or more of a contrast agent, blood, and vessel tissue; and the material (metallic or polymer) has a refractive index in a range of about 1.40 to 1.60, and preferably in a range between 1.47 to 1.57.

Certain imaging guidewires can also be configured to collect and detect auto-fluorescence light emitted by vessel tissue following irradiation with certain electromagnetic wavelengths, or to collect and detect fluorescence of applied fluorescent dies or agents that tag various tissues to fluoresce at known wavelengths. These imaging guidewires must be constructed with materials that themselves do not fluoresce in critical wavelength bands; otherwise, fluorescence of the window material could interfere with the efficient detection of low-level fluorescence from tissues. In addition, according to one or more embodiments of the present disclosure, one important aspect that can improve the imaging performance of the imaging guidewire 100 is the omission of rotation of the imaging core 200. Advantageously, non-rotating imaging core 200 is able to obtain OCT images free of NURD, as discussed later in more detail.

In the present application, metallic guidewire body using telescopic, concentric, and nested hypotubes allows for a guidewire design of minimum diameter due to tight dimensional tolerance control, and use of lubricants and/or low-friction coatings. However, alternative materials may also be used. Grilamid is a very common polymer used in the fabrication of catheter shafts, and could be used to create a guidewire window having telescopic, concentric, and nested hypotubes. In addition, the window section of the imaging guidewire may be made using other medical-grade thermoplastic polyurethane (TPU) and/or thermoplastic elastomer (TPE) materials, which are also applicable as tubing extrusion materials for medical devices that demand high precision and consistency. Commonly known catheter tubing materials may include acrylic, PVC, HDPE, Polyurethane, Polycarbonate, Nylon, PEBAX®, FEP, PFA, ETFE, PTFE (liners), PEEK, TPE, Grilamid®, PEBAX, ABS, among others.

### <Distal Tip of the Guidewire Body>

FIG. 6 shows in more detail the distal tip 160 and the atraumatic cap 170. As illustrated in FIG. 6, the distal tip 160 of the guidewire body is comprised of coiled wire 162, a core wire 161, and an atraumatic cap 170 (tip ball), shown at the right side of the drawing. On the left side of the drawing, a proximal bulbous element 163 of core wire 161 is locked inside support tube 141. More specifically, the distal tip 160 includes a coiled wire 162 and a metallic core wire 161, which are combined to form a “floppytip” at the distal end of the guidewire body. The imaging guidewire 100 is designed to provide optimal tip flexibility and effective torque transmission. To that end, the distal tip 160 of the guidewire body connects to a window support tube 141 via the metallic core wire 161, and connects to the window section 140 via coils of the coiled wire 162, which are embedded into the window wall material. The metallic core wire 161 includes a proximal bulbous element 163, a waist 165, and a distal element 164. The proximal bulbous element 163 is locked inside the window support tube 141 to provide additional tensile strength in the event of tip entanglement, so the whole device comes out in one piece. At the distal end of the floppy tip, the atraumatic cap 170 is a solid ball of platinum or iridium from melting the coil wire(s) that is welded to the metallic core wire 161.

According to one or more embodiments, the metallic core wire 161 and the coiled wire 162 that forms the floppy tip 16o can be made of platinum, or platinum mixed with a radio-opaque material such as iridium. The atraumatic cap 170 can also be made of radiopaque material by welding the platinum coiled wire 162 to the metallic core wire 161. The metallic core wire 161 can also be implemented as a nitinol or stainless steel core wire, and the coiled wire 162 can be a platinum or platinum-iridium coil. To enhance visibility under fluoroscopy, the coiled wire 162 that forms the floppy tip can be platinum, or in some cases, the coiled wire 162 can be made of 90% platinum doped with 10% Iridium oxide.

In alternate embodiments, the floppy tip metallic core wire 161 fits rotatably and slidably within a reduced inner diameter 141a of the support tube 141. In this manner, the overall assembly of core wire 161 and support tube 141 has a limiting element that rotatably and slidably locks the floppy tip core wire 161 to the support tube 141 to increase the tensile strength of the device. If entanglements occur during a procedure, the guidewire device could break and become inoperable in some cases. Therefore, to ensure patient safety, the guidewire body is locked to the floppy tip by the core wire 161 remaining locked with the support tube 141. In other words, the guidewire remains in one piece, and does not leave behind lose parts within the anatomy.

The floppy tip core wire 161, preferably made of nitinol and possibly stainless steel, tapers from around 0.007 inches diameter at the waist 165 to around 0.003 inches at the distal end to provide lateral support between the window section 140 and the floppy tip segment. In some embodiments, a thin, rectangular, wire can be welded to the tip of the core wire 161 to form the distal element 164. The rectangular wire can aid shapability of the distal tip for enhanced navigation purposes. Moving proximally, the floppy tip core wire 161 also tapers back down from the waist 165 of 0.007 inches to around 0.003 inches before terminating in the bulbous element 163. The bulbous element 163, which is formed by melting and welding the coil wire(s) to the core wire, locks the core wire 161 to the guidewire body. Lateral stiffness of the 0.003 inches diameter section of the floppy tip combines with the support tube 141 to roughly match the stiffness of adjacent sections of the guidewire.

The window section 140 must be as rigid as possible because the translating core leaves an unsupported space during imaging pullbacks. The floppy tip is formed by winding platinum wires into coiled wire 162, which are welded together and also welded to the core wire 161 to provide tensile strength for the floppy tip. In one embodiment, the coiled wire 162 is made of platinum or platinum-iridium for bright radio-opacity, while the core wire 161 is made of nitinol or stainless steel for tensile strength. The floppy tip provides an atraumatic cap on the guidewire that both aids in navigating through tortuous vessels while limiting potential damage to vessels that a rigid device would cause.

The floppy tip length is generally short to allow maximum lateral stiffness as distally as possible so that catheter devices guided by the guidewire can reach deeper into small vessels. At the same time, the floppy tip length should be long enough to provide effective navigational performance. The very distal, shapeable segment of the floppy tip can be about 1 to 2 cm long, with a 3 to 15 cm tapering stiffness segment just proximal to the shapeable segment for safety purposes because the guidewire body is quite stiff laterally, and could cause damage to a sensitive anatomy without a gradually softening tip. The gradual taper in lateral stiffness also enables the guidewire to selectively navigate through torturous vessels. Since guidewires are generally around 180 cm long, the distal tip 160 is proportionally only about 1/50^{th} to about 1/20^{th} of the entire length of the guidewire device.

In operation, as the imaging core is withdrawn, the window segment loses the support from the core that increased its lateral stiffness, leaving an unsupported section at the window area. To ensure that the guidewire body remains physically unchanged during and after imaging, the window support tube 141 extends distally from the imaging core to the distal tip. The window support tube 141 has either small holes or ‘window’ cut thru the wall and corresponding to the light beam emanating from the distal optics reflector, or, is substantially transparent to avoid the need of holes.

### <Guidewire Tapering Stiffness>

As noted above, it is desirable that imaging guidewire 100 is configured to provide stable torsional transmittance while providing gradual transition in lateral stiffness from the proximal end to the distal end of the guidewire body. More specifically, it is desirable to form a smooth and gradual transition from the proximal rigid hypotube section 121 to the more flexible distal sections to minimize kinking or buckling, and to more effectively transfer linear and torsional forces for navigating the distal tip of the guidewire from the proximal end thereof. In the present disclosure, to achieve smooth and gradual transition of lateral stiffness, each section of the guidewire is designed with specific attributes in terms of materials, durometer values, dimensions, aspect ratios, stiffness transition, etc., among other considerations.

It should be noted that, when a catheter or guidewire body is passed through a tortuous anatomy (e.g., from the femoral artery into the aorta, and then into coronary arteries), the guidewire body is elastically deformed so that its shape is adapted to the shape of the given anatomy. When advancing a guidewire though a vessel, the distal end of the wire can touch the vessel wall, and the forces acting on the guidewire can cause a deflection of the tip. For this reason, the forces acting on the vessel wall should be as small as possible to minimize the risk of damage to the vessel wall and/or to minimize the risk of buckling the instrument. Accordingly, it is necessary that guidewires should have suitable mechanical characteristics concerning flexural rigidity, torsional rigidity, tensile strength, as well as buckling and kinking resistance.

The present disclosure provides additional guidewire improvements by designing the guidewire with a non-rotatable imaging core to eliminate angular non-uniformities that cause NURD commonly attributed to rotating core designs. Another embodiment of the present disclosure includes designing the guidewire with a dual-drive rotation-by-translation imaging core to eliminate angular non-uniformities that cause NURD commonly attributed to single (proximally) driven rotating core designs. Either of these embodiments is further enhanced by designing the guidewire body with one or more stiffness-transition zones to appropriately control lateral stiffness during navigation.

### <Non-Rotatable Imaging Core>

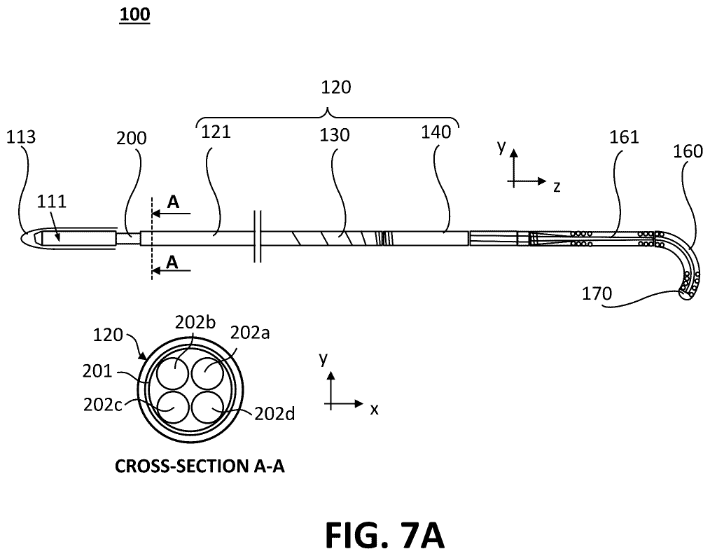

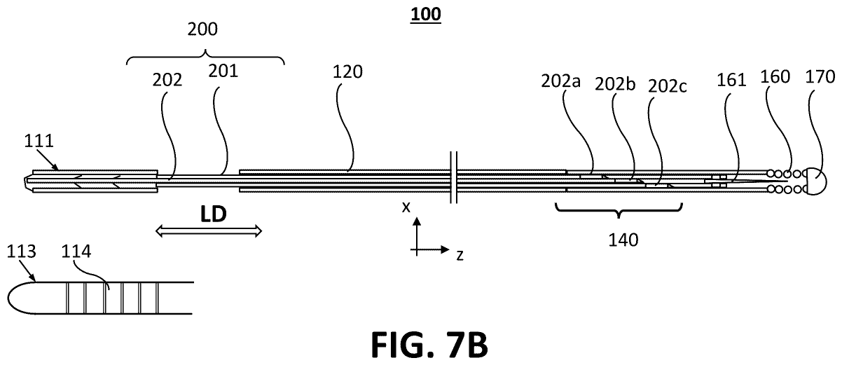

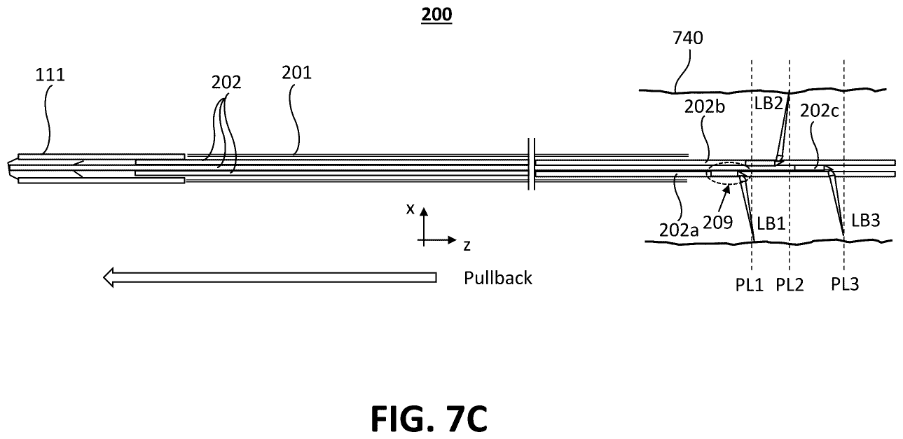

An imaging guidewire 100 with a non-rotatable imaging core 200 is described in more detail with reference to FIG. 7A through FIG. 10. According to this embodiment, the imaging guidewire 100 includes a guidewire body comprised of a proximal shaft section, a mid-shaft section and a distal shaft section. A shapeable or pre-shaped distal tip is attached to the distal shaft section. A rigid hypotube section 121 corresponds to the proximal shaft section, a semi-rigid slotted hypotube section 130 corresponds to the intermediate shaft section, a window section 140 corresponds to the distal shaft section, and a distal tip 160 corresponds to the shapeable or pre-shaped distal tip. In one embodiment, a novel solution to NURD and lack of rotational fidelity is provided by omitting rotation of the imaging core. Specifically, a non-rotatable imaging core 200, where the drive cable and rotation of the imaging core are omitted, provides more space for tailoring the wall thickness of the guidewire sections according to the desired stiffness of each section of the guidewire body, while ensuring kink resistance and roundness of the shaft. Since a drive cable is not used, a vessel wall is imaged by operating an imaging core 200 via a system that excites multiple fibers to scan the vessel wall without rotating the imaging core. This is accomplished by simultaneously or sequentially exciting each of a plurality of optical fibers arranged in the imaging core 200. This ensures that images are acquired without NURD using a novel guidewire body with a minimized diameter.

FIG. 7A, FIG. 7B, and FIG. 7C illustrate in more detail specific components of the imaging guidewire 100 and the non-rotatable imaging core 200. FIG. 7A is a side-view of imaging guidewire 100 along a y-z plane, as seen in the lengthwise direction. In a non-operating state, the imaging guidewire 100 has the proximal end thereof covered by a protective sleeve 113. FIG. 7B is a top-view of imaging guidewire 100 along an x-z plane. In FIG. 7B, the imaging guidewire 100 is shown with the protective sleeve 113 removed from the proximal end of the guidewire body. FIG. 7C is a schematic of the non-rotatable imaging core 200 without the guidewire body 120. In this embodiment, the imaging core 200 scans the vessel 740 with multiple light beams (LB1, LB2, LB3, etc.) which are orthogonal to each other, and also nearly orthogonal to the longitudinal axis Ox of the guidewire body.

In FIG. 7A, a cross-section A-A of the proximal connector 111 shows an axial view of the non-rotatable imaging core 200 on the x-y plane perpendicular to the longitudinal axis Ox of the guidewire body. The guidewire body 120 is formed of a hypotube (an outer hypotube), which is the main body segment of the imaging guidewire 100. The guidewire body 120 encloses the core hypotube 201 which in turn encloses a plurality of core optical fibers 202 (a plurality of fiber-optic waveguides). In the example shown by cross section A-A, the core hypotube 201 encloses a first core optical fiber 202a, a second core optical fiber 202b, a third core optical fiber 202c, and a fourth core optical fiber 202d. Depending on the diameter of the core optical fibers 202 and the diameter of the guidewire body, a large number of optical fibers can be arranged inside the inner diameter of the core hypotube 201. The proximal ends of core optical fibers 202 are held together by the proximal connector 111. In one embodiment, the proximal connector 111 is a modified fiberoptics ferrule having an outer diameter (OD) approximately equal to the diameter of the guidewire body 120 (see FIG. 3). In one prototype implementation, the proximal connector 111 is a modified ceramic ferrule having an OD of about 0.014 inches (0.3556 mm). In such embodiment, the proximal connector 111 is configured to hold the plurality of fibers 202 in a circular arrangement (see FIG. 3 and FIG. 8A). The plurality of core optical fibers 202, each having an outer diameter of 80 microns, is arranged in the lumen of core hypotube 201. In this embodiment, the core hypotube 201 has an inner diameter of about 0.0095 to 0.0135 inches (0.2413 to 0.3429 mm), or more preferably 0.0095 to 0.0105 inches (0.2413 to 0.2667 mm). From these exemplary dimensions, it can be understood that the guidewire body, and in particular the proximal connector 111 are delicate components that could be easily contaminated when swapping catheters during a procedure. For this reason, the protective sleeve 113 is used to keep the proximal connector 111 clean and dry until imaging use.

In one embodiment, the protective sleeve 113 is a thin-walled polymer-based shrink tube made from, for example, polyethylene terupthalate (PET). PET is a clear, strong, and lightweight plastic so that it can be heat-shrunk onto the proximal connector 111 such that the protective sleeve 113 stays tightly in place until manually removed by the user. At least a holding portion 114 of the protective sleeve 113 may be slotted or notched or knurled to facilitate easy handling by the user with gloved hands. The protective sleeve 113 can be reusable so that multiple catheters can be threaded over the proximal connector 111 without contaminating the proximal endfaces of the core optical fibers 202. In the event of the fibers becoming soiled, a solution of isopropyl alcohol (IPA) and water is used to flush off debris and blood contaminants that could interfere with imaging.

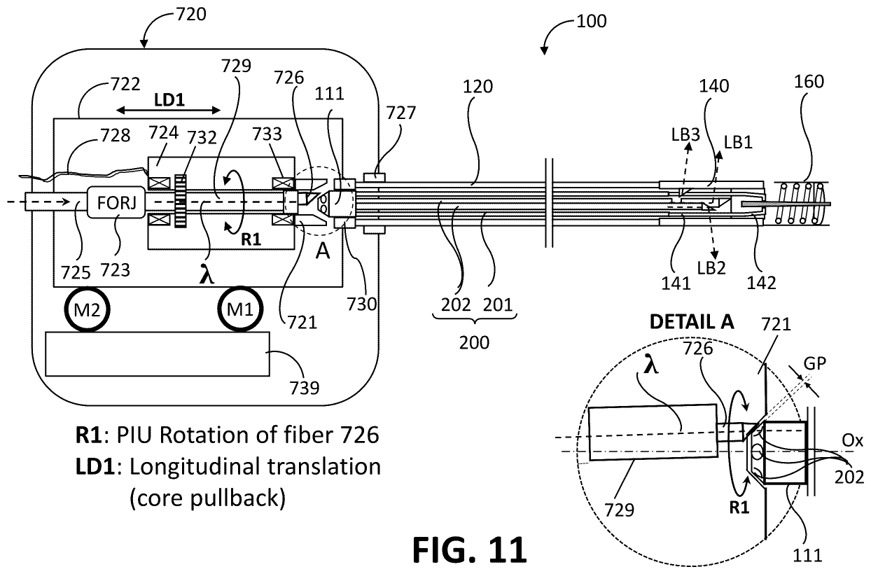

Prior to conducting a procedure, the protective sleeve 113 must be removed from the proximal end of the guidewire body to enable connection of the guidewire to PIU 720 for imaging. When the protective sleeve 113 is removed from the proximal end of the guidewire body, the proximal connector 111 becomes visible and exposed to the environment. When the proximal connector 111 is inserted into the PIU 720, a first gripping unit 727 grips the proximal end of the guidewire body 120, and a second, rotatable and/or translatable gripping unit 730 receives the proximal connector 111 and engages with the tapered outer surface 112 of the proximal connector 111 (see FIG. 11).

According to at least one embodiment, the proximal connector 111 is a modified ceramic ferrule custom-machined from a standard micro-miniature telecommunications fiber-optic connector that has been modified to suit the dimensions of the guidewire body. Some modifications include, but are not be limited to, forming a precision ground tapered outer surface 112, a precision ground chamfer 203 at the proximal end, and precision drilling of satellite holes (for each core fiber 202 of the non-rotating imaging core) positioned in a circle about the longitudinal axis of the lumen (see FIG. 8A).

The proximal connector 111 is not limited to machined ceramic ferrules. Ferrules made from any suitable material, such as metal, ceramic (Zirconia), glass, or molded polymer material may also be applicable. Ferrules applicable to proximal connector 111 may comprise substantially cylindrical portions, or portions comprising cylindrical shapes having channels in the cylindrical shape for introducing the core optical fibers 202. The ferrules applicable to proximal connector 111 are preferably cylindrical in shape configured to fit within the inner diameter of core hypotube 201. In some embodiments, ferrule diameters may be between about 0.01 to 0.015 inches, and in further embodiments, cylindrical portions of ferrules embodying features of the proximal connector may a diameter between about 0.013 to 0.014 inches. The proximal connector 111 has a tapered outer surface 112.

The rigid hypotube section 121 is nested around the proximal portion of core hypotube 201 to reinforce the OD and stiffen the proximal shaft section of the guidewire body 120 while maintaining a minimum diameter. As shown in FIG. 2, FIG. 3, and FIG. 7B, the proximal end of the guidewire body 120 and the proximal end of the core hypotube 201 can be slidably joined to each other as nested tubes (one inside the other), and the nested hypotubes are capable of sliding with respect to each other in a lengthwise direction LD.

FIG. 7C shows the imaging core 200 irradiating a vessel 740 with a plurality of light beams each emitted from a separate core optical fiber 202. According to FIG. 7C, the non-rotatable imaging core 200 is configured to scan the vessel 740 with multiple orthogonal light beams. In FIG. 7C, distal ends of core fibers 202 are staggered for ease of illustration. However, in at least some embodiments, the distal ends of core optical fibers 202 can be arranged at a same plane as shown in FIG. 4B. The plurality of light beams can be emitted simultaneously or sequentially. Sequential excitation of each core fiber 202 positioned about the periphery of the proximal connector 111 can be considered as the mode of excitation most similar to the manner in which conventional OCT systems operate. A notable difference with respect to conventional OCT is that, while a conventional OCT system requires a torque transferring component (torque cable) to rotate the imaging core, in the present embodiment the distal end of each core optical fiber 202 transmits and collects light from/to the vessel wall without rotation of the imaging core.

To efficiently direct light onto, and collect light from, the vessel 740, each fiber 202 includes a distal optics assembly 209 at the distal end thereof. The distal optics assembly 209 includes at least a beam shaping component such as a lens, and also a beam directing component fused to, or grounded and polished on, the distal end of each beam shaping component. The beam directing component may include an angle-polished surface (e.g., a mirror) or a grating or a prism configured for directing and focusing a beam of light (LB) at the vessel 740. In some embodiments, a distal end of each core optical fiber 202 can be fused directly to a focusing lens (GRIN lens or ball lens), and provided with a prism or an angle polished cylindrical surface. A cylindrical surface advantageously focuses the light beam in one axis to prevent astigmatism caused by the cylindrical window outer surface. The use of fused distal optics at the distal end of each fiber is advantageous because there is no bonding that leaves glue in the joint that might attenuate light and degrade optical performance or physical strength. In other embodiments, each fiber 202 is provided with a molded lens, or with a lens-reflector combination attached to, polished on, or aligned with, the fiber end. In at least some embodiments, the core optical fibers 202 used in the non-rotatable imaging core 200 can include polarization maintaining (PM) fibers. In one embodiment, the simplest configuration for guiding the light from the imaging core to the vessel wall is a bare optical fiber having a distal end with a polished convex surface with different radii of curvature in the x-axis versus the y-axis to effectively focus the light emanating from the fiber and passing through the window into a beam on the vessel wall. But, to more efficiently collect the reflected and/or backscattered light, a beam-shaping component can fused or spliced directly to the distal end of each fiber 202.

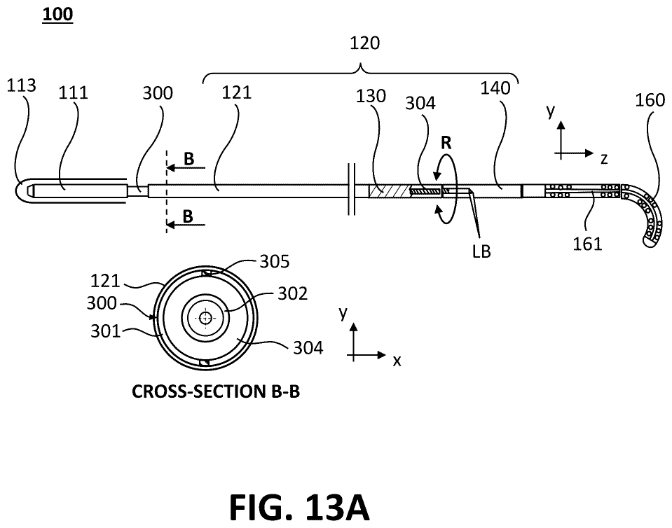

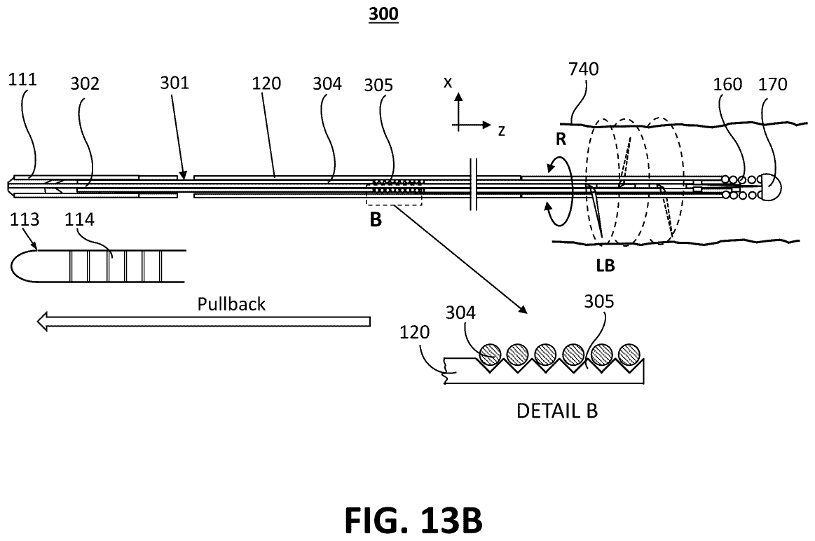

Regardless of the configuration of the optical fibers within the non-rotatable imaging core 200, the distal optics assembly 209 of each core optical fiber 202 is configured to direct a beam of light in an angular direction with respect to guidewire axis Ox. For example, as shown in FIG. 7C, the first fiber 202a may irradiate a first light beam LB1 in a first direction at a first plane PL1, the second fiber 202b may irradiate a second light beam LB2 in a second direction at a second plane PL2, the third fiber 202c may irradiate a third light beam LB3 in a third direction at a third plane PL3, etc. As the non-rotatable imaging core 200 is withdrawn (pullback) each light beam forms a line signal along the length of the vessel 740. Line signals from each fiber 202 are collected by the imaging core 200, delivered to the console 701, and processed to generate a basic wireframe image, as shown in FIG. 13, for example.

FIG. 8A and FIG. 8B show various views of the fiber-optic arrangement within the proximal connector 111 at the proximal end the imaging core 200. FIG. 8A is a 3D illustration that shows a back view of the proximal connector 111 of imaging guidewire 100 without the protective sleeve 113. FIG. 8B is a 2D back view that shows the proximal end (or a cross-section) of the proximal connector 111 enclosing a plurality of core optical fibers 202a, 202b, 202c, 202d.

The proximal connector 111 holds the plurality of core optical fibers 202 fixedly arranged in a non-rotatable shaft (core hypotube 201) which is slidably insertable into the guidewire body 120, which is the main guidewire body (also referred to as the outer hypotube). The proximal connector 111 may be a modified fiber connector ferrule connected to, or mounted on, the proximal end of the core hypotube 201. In the proximal connector 111, the fiber ends are polished at a small angle β (in a range of about 8 to 15 degrees) to minimize reflections. In FIG. 8A, the distal end of each of core optical fibers 202 is not parallel to the x-y plane. Instead, each fiber 202 has its proximal end polished at an angle β with respect to normal to a central axis Cx of the proximal connector 111.

In FIG. 8A and FIG. 8B, the imaging core 200 is shown to have four core optical fibers 202a, 202b, 202c and 202d arranged radially inside the proximal connector 111. At the proximal end of proximal connector 111 the modified ferrule has a chamfer 203, and the body of the proximal connector has a tapered outer surface 112, which facilitates easy insertion and precise alignment of the proximal connector with a receiving bore of the PIU 720. Specifically, a receiving shaft of the PIU 720 has a complimentary conical shaped bore like a Morse Taper that guides and precisely aligns the proximal connector 111 with the axis of rotation within the PIU rotation unit. The tapered outer surface 112 has a very small taper angle θ (theta) of about 5 to 10 degrees, and preferably between 5 and 8 degrees, with respect to the outer cylindrical surface of the proximal connector 111. This means that the proximal connector 111 tapers from right to left (in FIG. 8A), like a Morse Taper. In fact, all embodiments of the imaging core may include a proximal connector 111 with this precise tapered outer surface 112 to effect repeatable optical coupling with the exciting fiber, which rotates, orbiting the connecting fiber face around the axis of rotation within the PIU as the imaging core is pulled back.

Within the proximal connector 111, the core optical fibers 202a-202d are terminated at a pseudo angle β, which is around 7-12 degrees from normal to the axis of rotation, and formed by polishing all fibers simultaneously at an angle forming a conical shaped surface at the proximal end, and the core fibers are arranged radially within (inside) the proximal connector 111. As stated elsewhere, the proximal connector 111 is a modified ferrule with either a single inner diameter to fit all core fibers, or with a hole or outer groove for each core fiber. The fiber end faces are actually slightly rounded due to all fibers being polished at the same time while rotating the core. Alternatively, the proximal connector 111 may be provided with a single inner diameter formed to snugly fit the desired number of optical fibers. In the arrangements illustrated by FIG. 2, FIG. 3, FIG. 7A (cross-section A-A) and FIG. 8A-FIG. 8B, the imaging core 200 spans from the proximal connector 111 through the rigid hypotube section 121, through the slotted hypotube section 130, and to the window section 140 extending the majority of the length of the guidewire body, but not reaching the distal tip 160. In FIG. 8A-FIG. 8B, the proximal connector 111 is shown to have four optical fibers, for example, arranged concentrically around the central axis Cx. Naturally, FIG. 8A and FIG. 8B are mere examples of the manner in which the proximal connector 111 can hold the proximal ends of core optical fibers 202. Other embodiments with different number of fibers and different arrangements of the fiber ends within the proximal connector 111 are contemplated. For example, core optical fibers can be arranged in a ribbon-like arrangement, such as described in U.S. patent Ser. No. 10/578,798, which is incorporated by reference herein in its entirety.

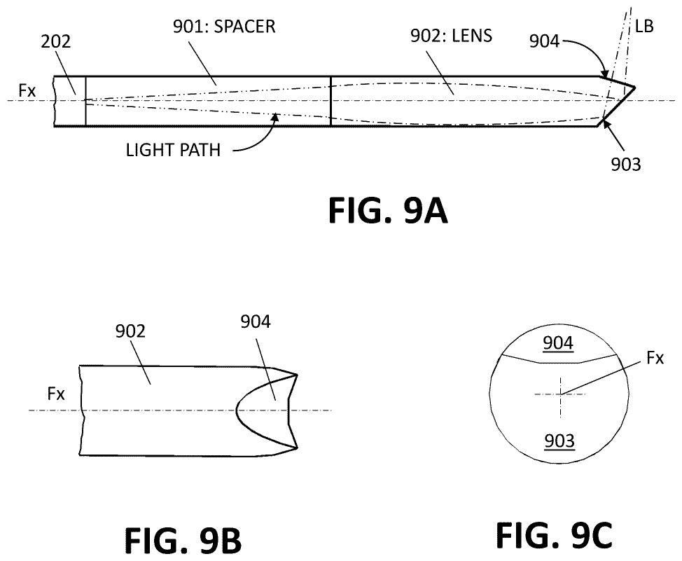

FIG. 9A, FIG. 9B, and FIG. 9C illustrate various views of distal optics assembly 209. FIG. 9A shows a side view of the distal optics assembly 209. The distal optics assembly 209 includes a distal end of core optical fiber 202, spacer 901, a GRIN lens 902, a reflector surface 903, and a cylindrical surface 904. FIG. 9B shows a top view of the GRIN lens 902 with the cylindrical surface 904 having a concave shape. FIG. 9C shows a front view of the reflector surface 903 and the cylindrical surface 904. As evidenced from these drawings, the spacer 902 is a cylindrical component, which can be implemented by a short segment of coreless optical fiber having a fiber axis Fx. The reflector surface 903 is a flat surface formed at an angle with respect to the fiber axis Fx. The reflector surface 903 may be a flat surface having an angle to meet the requirement of total internal reflection (TIR), or may be a flat surface with a reflective coating that forms a mirror surface. The cylindrical surface 904 can be configured to focus the light beam more in a first direction than in a second direction perpendicular to the first direction. For example, each fiber 202 can be polished with a cylindrical surface 904 to focus the emanating radiation beam into a beam that is focused more in one axis than another to compensate for lensing effects from the cylindrical window section 140. The distal optics assembly 209 can be applicable to the core optical fibers 202 arranged in the imaging core 200, as well as to core optical fibers 302 of imaging core 300. The distal optics assembly 209 can be achieved by alternate construction methods that could include an injection molded lens component and/or spacer component that can meet the desired functionality of appropriately focusing a light beam (LB) on a vessel wall.

FIG. 10 shows a conceptual illustration of a non-rotatable imaging core 200 configured to acquire image data from a vessel 740 using orthogonally irradiated beams of radiative energy, where all light beams are equally spaced around the interior of the vessel 740. It will be understood that a lumen of a vessel 740 may have an irregular (non-circular) inner surface, but for purposes of illustration, FIG. 10 shows a circular lumen. The imaging guidewire 100 is inserted through a guide catheter, into the vessel of interest, with the imaging plane advanced into the lumen and positioned just beyond the area of interest within the vessel. Once in place, the system controls the imaging core 200 to irradiate each section (sector) of the lumen with a separate core fiber 202 through the transparent material of window section 140. For example, as shown in FIG. 10, a first core fiber 202a irradiates a light beam LB1 towards a first imaging sector (IS1), a second fiber 202b irradiates a line beam LB2 towards a second imaging sector (IS2), a third fiber 202c irradiates a line beam LB3 towards a third imaging sector (IS3), a fourth fiber 202d irradiates a line beam LB4 towards a fourth imaging sector (IS4), etc.

It should be understood that FIG. 10 shows a front view of the distal optics assembly 209 of each fiber. As such, the light beams LB1, LB2, LB3, and LB4 may irradiate sectors of the vessel wall that are at slightly different planes (staggered in the lengthwise direction). More specifically, depending of the parameters of the cylindrical surface 904 of the distal optics assembly 209, each core optical fiber 202 can form a beam shape to optimize imaging according to desired outcome. In addition, while light beams are substantially orthogonal to each other, all light beams are projected at an angle in a range of 4 to 25 degrees with respect to the normal to the device axis to minimize unwanted effects (backreflection) from the inner surface of the window.

In operation, the imaging guidewire 100 initially acts solely as a regular guidewire, except during withdrawal (pullback) when the imaging core is withdrawn about 50 mm while scanning the vessel wall with light beams. During the scanning, image signals are continuously recorded. More specifically, initially the imaging guidewire 100 is inserted into a vessel as a regular guidewire. When the imaging plane of the imaging guidewire 100 is beyond the stenosis of interest, the guidewire is placed in a ‘parked position’; then, the protective sleeve 113 is removed from the guidewire, and the proximal connector 111 is inserted into the PIU 720 to connect the imaging guidewire to the FORJ, as explained in more detail below. The OCT system is calibrated for each individual guidewire, and imaging is accomplished by retracting the imaging core 200 while the PIU excites the core optical fibers 202 of the guidewire either sequentially or simultaneously. A basic outline of the vessel inner diameter (ID) can be reconstructed as the imaging core 200 is retracted (pullback) and each fiber 202 of the imaging core 200 irradiates an angular portion (sector) of the vessel wall simultaneously, or in rapid succession. Since each beam traces the profile of a vessel wall section, profile data can be combined into a wireframe for 3D reconstruction. The reconstructed wireframe data can be enhanced via animation to look more like an active anatomical lumen. In one example, the composite reconstructed image data can include OCT data obtained from light collected through the fiber core, and then combined with tissue fluorescence data obtained from fluorescence light collected through the 1^{st} (inner) layer of cladding of each fiber. These data obtained with the guidewire can also be combined with fluoroscopy or CT imaging data.

### <Connection of Imaging Guidewire Having Non-Rotatable Imaging Core to PIU>

FIG. 11 illustrates an embodiment of a PIU 720 configured to connect the imaging guidewire 100 having a non-rotatable imaging core 200 to the system console 701. The PIU receives light from one or more light sources (e.g., an OCT light source and fluorescence excitation light source) via a source optical fiber 725. The PIU 720 includes a fiber optic rotary joint (FORJ) 723, a rotary unit 724, and a pullback unit 722. The FORJ 723 includes optics for transmitting light from an OCT light source and light from an excitation light source, and for guiding the combined light through a connecting fiber 726 and/or a fiber connector 729 towards the proximal connector 111. That is, the connecting fiber 726 and/or fiber connector 729 connect the source fiber 725 to the core optical fibers 202, and vice versa. The rotary unit 724 includes a torque source, such as a rotational motor. The pullback unit 722 includes, for example, a platform 739. The platform 739 includes one or more motors M1-M2 that move a single-axis translation stage, or includes a piezoelectric-actuated linear stage. The rotary unit 724 includes, for example, a hollow shaft motor configured to rotate, orbit, or oscillate the connecting fiber 726 and/or the fiber connector 729 with respect to the proximal connector 111. An encoder 732 or a rotational sensor (e.g., Hall-effect sensor) is provided with the rotary unit 724 to track the rotation of connecting fiber 726 and/or the rotation of fiber connector 729 so that the system can identify which core fiber is currently being excited. One or more high-precision bearings 733 are provided on the rotary unit 724 to ensure optimal rotation of the connecting fiber 726 and/or fiber connector 729. A centering unit 721 is a receiving shaft directly connected to the rotary unit 724 (e.g., the hollow shaft motor), and the centering unit 721 is configured to hold the fiber connector 729 at a predetermined position and/or orientation with respect to a rotational axis of the rotary unit 724. In one embodiment, the centering unit 721 holds the fiber connector 729 offset with respect to the rotational axis of the hollow shaft motor. Offsetting the fiber connector 729 with respect to the rotational axis of the PIU enables the fiber connector 729 to rotate or oscillate in direction R1 in an orbital path keeping the connecting fiber face close to core fiber faces during operation as the fiber connector 729 sequentially excites each core fiber 202 one at a time. In FIG. 11, DETAIL A shows the connecting fiber 726 transmits light of wavelength (lambda) along a path offset with respect to the guidewire central axis Ox.

The source optical fiber 725 delivers radiative energy (light of an appropriate wavelength λ (lambda)) from the console 701 to the FORJ. The FORJ 723 guides the light from the source optical fiber 725 to the imaging core 200 via the connecting fiber 726 and the fiber connector 729. The centering unit 721 is a short hallow shaft with a simple conical shape that receives and aligns the proximal connector 111 to be collinear with the center of rotation in the PIU. The pullback unit 722 holds the rotary unit 724 collinear with a first gripping unit 727 that prevents the guidewire body 120 from moving as the imaging core 200 is pulled back into the PIU. During pullback, the rotary unit 724 holds the connecting fiber 726 and/or fiber connector 729 offset with respect to the longitudinal axis Ox of guidewire body. The first gripping unit 727 engages with the proximal end of guidewire body 120 (i.e., the outer hypotube), and holds the guidewire body 120 without movement, while allowing only linear movement of the imaging core 200, in this embodiment. A second gripping unit 730 engages with the conical outer surface 112 (see FIG. 8A) of the proximal connector 111. The second gripping unit 730 includes a receiving bore fixedly mounted on the pullback unit 722.

Electrical connections, such as a cable bundle 728, carry control signals and electrical power from the system console 701 through the umbilical 710 to the PIU 720 to operate the non-rotating imaging core 200 for acquiring an image of a given lumen (e.g., vessel 740 shown in FIG. 1 or FIG. 7C). The source fiber 725 and cable bundle 728 are arranged along the umbilical 710 (shown FIG. 1).

Prior to a procedure, when the proximal end of guidewire body is inserted into PIU 720, the first gripping portion 727 grips and locks the guidewire body 120 (the outer hypotube). Then, the receiving bore of the second gripping portion 730 receives the proximal connector 111, and engages with the tapered outer surface 112. The receiving unit 721 has an inclined surface, which causes the chamfer 203 of proximal connector ill to keep the proximal connector aligned substantially parallel to the rotation axis of the rotary unit 724. In operation, the second gripping portion 730 pulls back the imaging core 200 (to the left of the image), while the first gripping portion 727 holds the guidewire body 120 stationary. The guidewire body 120, which forms the main body of the imaging guidewire 100, is in contact with the anatomy of the patient inside the patient's arteries, and as such the guidewire body 120 cannot move during imaging. The PIU 720 must ensure that guidewire body 120 remains stationary, while pulling back the imaging core 200 to enable the distal optics of each core fiber 202 to direct the excitation light beams LB1, LB2, LB3, etc., at the wall of the vessel.

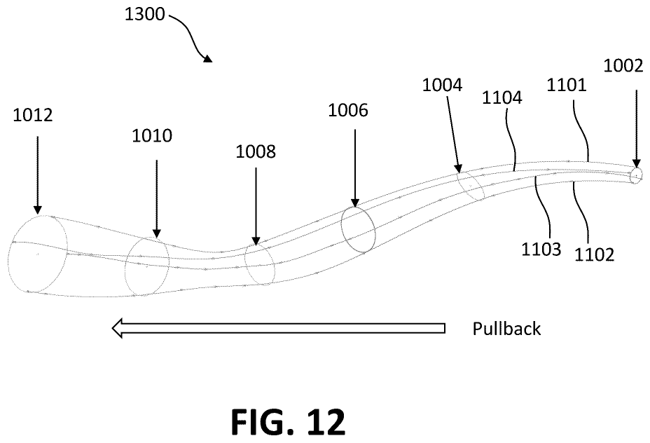

Inside the PIU 720, the fiber connector 729 maintains a small distance (a gap GP) of about 0.50 to 25 microns between the connecting fiber 726 and the proximal ends of core fibers 202 of the non-rotatable imaging core 200. The gap GP between the end face of connecting fiber 726 on the PIU-side and the fiber ends of core fibers 202 on the imaging core side allows rotation by the PIU of the fiber connector 729 without causing friction or damage of connecting fiber 726 against the fiber ends of the non-rotating imaging core. The gap GP can be an air gap, or it can be optionally filled with refractive index-matching materials (e.g., a gel or lubricant) to minimize optical reflections. Since the proximal end of the imaging core 200 is inserted into the PIU, the gap GP (either air filled or refractive index-matching material filled) is formed inside the PUI, and is not visible to the user. The rotary unit 724 of PIU 720 rotates the connecting fiber 726 and/or fiber connector 729 to sequentially excite each of the core optical fibers 202 one at a time. The imaging core 200, upon receiving light from the fiber 726 to each core optical fiber, emits a plurality of light beams sequentially in directions nearly orthogonal to the guidewire body, e.g., as shown in FIG. 7C and FIG. 10. In this manner, the guidewire 100 can scan the vessel 740 without any rotation of the imaging core 200, which is advantageous in avoiding NURD. In addition, the imaging core 200 can be withdrawn (pullback) without rotation, while scanning the vessel wall the plurality of light beams, thereby creating a wire-frame image of the vessel 740 (see example shown in FIG. 12).

The novel arrangement of a non-rotatable imaging core 200 provides one or more notable advantages to the guidewire properties due the fact that no rotating parts are used to acquire image data. Imaging beams are delivered to the vessel 740 via individual fibers which are excited by a single offset fiber or fiber connector of the PIU. The single offset fiber or fiber connector of the PIU rotates at high speed to deliver radiation (light) of appropriate wavelength to each core fiber 202 in rapid sequential fashion. The encoder 732 is provided with the rotary unit 724 to track rotation of the connecting fiber 726 and/or fiber connector 729. The system can use a signal from encoder 732 to determine which core fiber 202 is currently being excited, and how to arrange the data collected from each fiber 202 to generate the vessel image.

In the PIU 720, the centering unit 721 may include an electronic-turret that rotates the PIU-side connecting fiber 726 and fiber connector 729 to excite the plurality of core optical fibers 202 (e.g., four to forty eight) separately, one at a time. In this embodiment, the system is configured to collect light from each separate core fiber 202, and to reconstruct an image by combining the line data from each fiber into a vessel reconstruction or wire frame image. To distinguish which fiber each line signal is received from, polarization multiplexing is possible to implement by using polarization-maintaining (PM) fibers. In one prototype embodiment, the inventor has implemented an imaging core having four to forty eight core optical fibers 202 each having a diameter of 80 microns. Time domain multiplexing can also be implemented by using optical fibers with different fiber lengths, or by providing an optical delay line to vary the optical path length of each fiber by a differentiable length.

An OCT system employing only one interferometer can differentiate light coming from a plurality of optical fibers in several ways as described above. An alternate embodiment of the non-rotatable imaging core 200 can be implemented where excitation of all core optical fibers 202 of the imaging core occurs simultaneously by a plurality of excitation fibers (e.g., using a plurality of interferometers). The simultaneous use of multiple interferometers in an OCT system, one interferometer for each fiber of the imaging core, can be implemented to simultaneously collect light at a plurality of longitudinal locations during a pullback without rotating the imaging core. Other novel methods of differentiating light received sequentially or simultaneously from multiple fibers are possible. Some examples include the use of polarization multiplexing techniques where each fiber is a polarization-maintaining (PM) fiber. In polarization multiplexing, the polarization orientation from each PM fiber is matched to polarized beam splitters or other polarization-detecting devices to tell the system where the image data is to be positioned in the image. An example of polarization multiplexing is discussed in pre-grant patent application publication US 2007/0015969 A1, which is incorporated by reference herein for all purposes. Another proposed method of differentiating signals received from separate fibers is via time domain multiplexing, where individual fibers vary in length.

During pullback, the rotary unit 724 (e.g., a rotary motor) rotates the connecting fiber 726 and/or the fiber connector 729 so that light is transmitted in rapid succession to the core optical fibers 202 arranged in the proximal connector 111 of the imaging core 200. The rotation of the PIU rotary unit 724 can be up to 200 Hz, but if necessary the system can reduce the time required for pullback. Because the imaging core 200 is positively engaged with the pullback unit 722 via the receiving bore of the second gripping portion 730 engaging with the tapered outer surface 112, any given change in longitudinal position corresponds to a longitudinal movement (translation) of each light beam in the longitudinal direction (LD1). So because the PIU knows where the imaging core 200 is longitudinally with respect to the beginning of pullback, the system can calculate where the beam is pointing, and can form an image of the vessel by combining signals corresponding to light reflected or backscattered from the vessel wall and collected by each optical fiber, as illustrated in FIG. 12.

### <Image Reconstruction for OCT Data Acquired by Non-Rotatable Imaging Core>

In general, the imaging guidewire can be applicable to imaging techniques that may provide real-time 2D or 3D imaging of a bodily lumen, in particular a vessel. FIG. 12 illustrates an example of a wireframe image 1200 reconstructed from image data collected by the non-rotatable imaging core 200 of imaging guidewire 100. In this embodiment, a signal for each radiation beam that scans the vessel 740 can be collected by the distal optics assembly 209 of each core optical fiber. The collected signals are detected and processed by one or more processors for image reconstruction using well-known techniques including, for example, Time Domain Multiplexing (TDM). The wireframe image 1200 can be displayed in a longitudinal or L-mode format. In one embodiment, a pullback imaging operation of a non-rotatable imaging core 200 having four optical fibers can generate the wireframe image 1300 shown in FIG. 12. OCT image data collected by the imaging guidewire 100 includes a first line 1101, second line 1102, a third line 1103, a fourth line 1104, each corresponding to irradiation and collection from/to a separate core optical fiber 202 included in the imaging core 200. These lines can be combined with image data from a secondary modality (e.g., fluoroscopy) to show regions of the vessel 140 that are good candidates for stent deployment based on stenosis, and/or to show regions of the vessel to avoid stenting (e.g., side branches). For example, in FIG. 13, the system can correlate the image data collected by each optical fiber of the imaging guidewire (i.e., lines 1101, 1102, 1103, and 1104) with angiography or data from other imaging modality to show candidate positions for stent deployment. To that end, a cross-section (diameter) of the vessel may be acquired at various points along the pullback path by calculating the lumen area between the longitudinal data lines. For example, lumen areas of the vessel cross-section can be acquired at various positions (e.g., at a first plane 1002, second plane 1004, third plane 1006, fourth plane 1008, fifth plane 1010, sixth 1012, etc.) along the pullback path. Based on the lumen areas calculated from the OCT data at each plane, and correlating the lumen areas with fluoroscopy data, the location for stent deployment and/or the level of stent expansion can be determined with high accuracy, without needing an additional imaging catheter. These types of investigations using OCT data co-registered with data of a secondary modality (e.g., angiography data, CT data, fluorescence data, etc.) can be used on one or more of pre-stenting, stenting, or post-stent follow-up procedures.

### <Dual-Drive Pseudo-Rotatable Imaging Core>

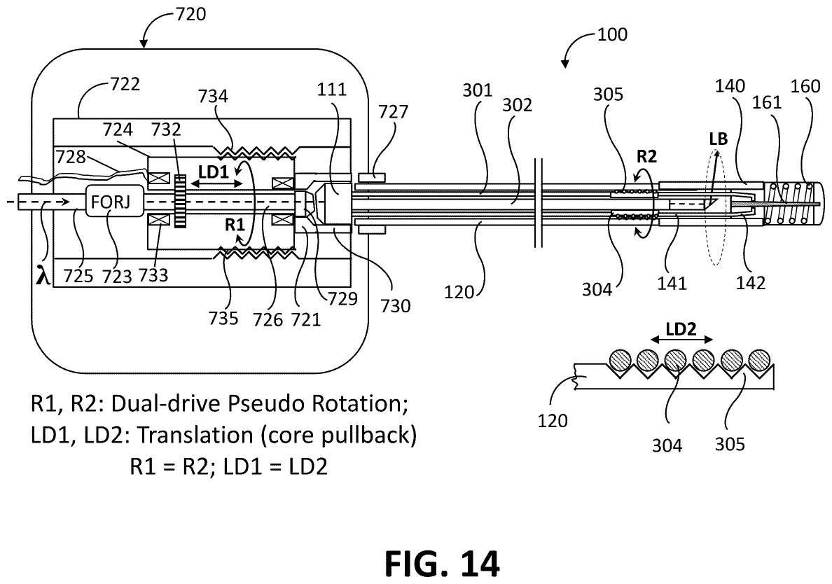

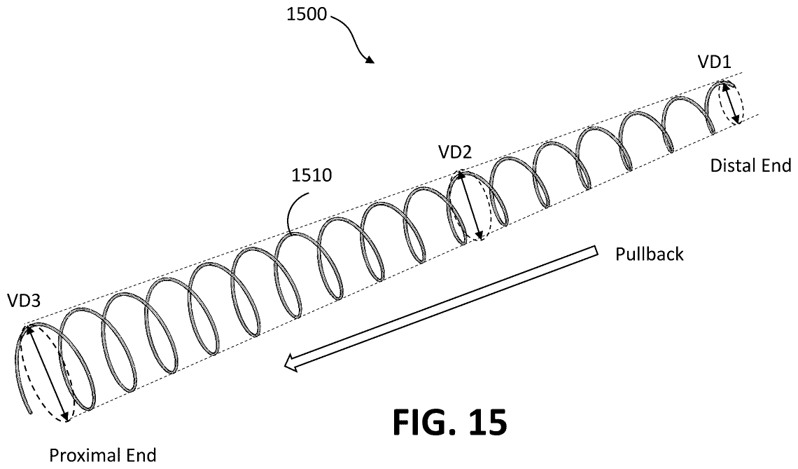

An imaging guidewire 100 having an imaging core 300 controlled by a rotation-by-translation scheme is described with reference FIG. 13A, FIG. 13B, FIG. 14, and FIG. 15. In this embodiment, the imaging guidewire 100 is comprised of a guidewire body identical to that of the previous embodiment, but the imaging core 300 is different from imaging core 200. According to this embodiment, the imaging core 300 is operated by a dual-drive rotation-by-translation scheme. The present embodiment provides an imaging guidewire 100 having a guidewire body with optimized mid-weight lateral stiffness and smooth stiffness transitions zones to avoid kinking, and a novel dual-drive rotation-by-translation rotary system which enables imaging of a vessel without NURD artifacts. The imaging guidewire 100 is configured to acquire accurate OCT images in short time with reduced image data, and without image artifacts. The same imaging guidewire can also be used in a NIRAF-OCT system to acquire co-registered OCT and fluorescence images of a vessel region.

FIG. 13A is a side-view of imaging guidewire 100 along a y-z plane in the lengthwise direction of the guidewire body. According to this embodiment, the imaging guidewire 100 includes a guidewire body 120 comprised of a proximal shaft section, an intermediate shaft section and a distal shaft section, and a shapeable or pre-shaped distal tip attached to the distal shaft section. Nested hypotubes form the majority of the guidewire body 120. The guidewire body 120 is comprised of an outer hypotube, a window section made of substantially transparent material, a distal tip. The outer hypotube includes a rigid hypotube section 121 that corresponds to the proximal shaft section and a semi-rigid slotted hypotube section 130 that corresponds to the intermediate shaft section. A window section 140 that corresponds to the distal shaft section, and a distal tip 160 attached distally to the window section 140 corresponds to the shapeable or pre-shaped distal tip.

In FIG. 13A, the imaging guidewire 100 is shown with the proximal connector 111 slightly pulled back, to show the imaging core 300, and the proximal connector is covered by a protective sleeve 113. The distal tip 160 is shown in a curved shape. The guidewire body 120 and distal tip 160 of imaging guidewire 100 are structurally similar to the same components shown and described with reference to the previous embodiment. In particular, similar to the previous embodiment, the rigid hyptube section 121, the semi-rigid slotted hypotube section 130, the window section 140, and the floppy tip 160 are configured to provide good torsional control and smooth stiffness transitions. Inside the guidewire body (i.e., within the central lumen), the imaging core 300 is arranged along the inner surface of the outer hypotube extending through the proximal shaft section to the mid-shaft section and extending into the distal shaft section. In the present embodiment, a novel dual-drive rotation-by-translation rotation system provides a pseudo rotation of a limited number of revolutions with high angular fidelity for accurate OCT imaging, while eliminating or significantly reducing non-uniform rotational distortion (NURD).

In FIG. 13A, a cross-section B-B shows an axial view of the pseudo rotatable imaging core 300 inside the guidewire body 120, as seen on the x-y plane perpendicular to the lengthwise direction of the guidewire body. The guidewire body 120 encloses a core hypotube 301, and the core hypotube 301 is configured to move telescopically (to translate linearly) with respect to the guidewire body 120. In the schematic example shown in FIG. 13A, the distal portion on the right side of the foreshortening lines is shown in cross-sectional view for ease of illustration. In addition, the cross section B-B shows the core hypotube 301 is directly affixed to the drive cable 304 via affixing means 305 such as welding, solder, brazing, glue, crimping, bonding, interference fit, etc., such that both structures (drive cable 304 and hypotube 301) enclose a single core optical fiber 302. Other affixing methods well known to persons skilled in the art are also contemplated for affixing the core hypotube 301 to the drive cable 304.

In the present embodiment, which is similar to the previous embodiment, the proximal connector in can be a modified ceramic ferrule mounted (attached) at the proximal end of the core hypotube 301. The proximal connector 111 can be attached to the core hypotube 301 by attaching means such as adhesives, melt-bonding, laser welding, soldering, etc. Unlike the previous embodiment, the proximal connector 111 of the pseudo rotatable imaging core 300 holds a single core optical fiber 302 substantially concentric with the longitudinal axis Ox of the guidewire lumen. In an implementation, the core optical fiber 302 is a multi-clad fiber (e.g., a double clad fiber (DCF)) arranged inside the drive cable 304; and the drive cable 304 is attached to the core hypotube 301. The core hypotube 301 may have an outer diameter of about 0.010 to 0.0135 inches, and the drive cable 304 is configured to be attached to, and to have roughly the same diameter as, the core hypotube 301, so as to be substantially concentric thereto. On the proximal end of the guidewire body 304, the protective sleeve 113 keeps the tapered outer surface 112 of the proximal connector 111 clean until use of the system. Along the length of the guidewire body, the core hypotube 301 is attached (e.g., welded of fused) to the drive cable 304 at least at one location (e.g., at the proximal end and/or near the window section). In this manner, the core hypotube 301 and drive cable 304 transfer torque from the PIU 720 to the distal end of the imaging core 300 with high angular fidelity.

In this embodiment, to maintain high angular fidelity of torque transmission from the proximal end to the distal end of the guidewire body, the guidewire body 120 (i.e., the outer hypotube) includes an inner threaded section 305 that closely matches an outer profile of the drive cable 304. As it will be appreciated by those of ordinary skill in the art, a drive cable, also referred to as torque cable or torque coil, is a highly flexible tubular shaft consisting of one or more layers of one or more thin wires coiled helicoidally to form a hollow shaft that provides torqueability, pushability, and kink resistance. In the present embodiment, the guidewire body 120 includes an inner threaded section 305 that engages with the outer profile of the drive cable 304, or a custom fabricated element with more desirable geometric and flexural properties, to provide positive positioning of the imaging core 300 during an imaging procedure. Specifically, when connected to the PIU 720, the imaging core 300 is pulled back to acquire image data by scanning the vessel 740, as described above with respect to the previous embodiment. During pullback, the coiled wire of the drive cable 304 follows along groves or teeth of the inner threaded section 305 of the guidewire body 120 while simultaneously being pulled back and rotated by the PIU with an identical angular rotation. This causes the imaging core 300 to rotate and translate inside the guidewire body 120, which remains stationary. The rotational effect caused by the dual drive element 306 of the core assembly 300 following along groves or teeth of the inner threaded section 305 of the guidewire body 120 is referred herein as a pseudo rotation, dual drive or a rotation-by-translation scheme. In other words, the imaging core 300 is not only actively rotated as in conventional OCT systems, but also the pseudo rotation is a result of the pullback force causing the coiled wire of the drive cable 304 or drive element 306 to follow along helical groves or teeth of the inner threaded section 305 of the guidewire body 120. In this manner, the optical fiber 302 scans the vessel 740 with a single light beam LB irradiated in a helicoidally moving path. The helicoidally moving path of the light beam LB follows a path determined by helicoidal outer profile of the drive cable 304 or drive element 306. FIG. 15 shows an example of an image acquired by imaging guidewire 100 equipped with the imaging guidewire 300 controlled by the rotation-by-translation scheme. Another form of pseudo rotation occurs during imaging pullback, where the connecting fiber 726 rotates and orbits about the axis of rotation in the PIU, sequentially exciting each core fiber 202 one at a time, simulating a rotating core.

FIG. 13B shows a side view of the rotatable imaging core 300, as seen in the lengthwise direction along the x-z plane. In this embodiment, to connect the guidewire body to the PIU 720, the protective sleeve 113 must be removed from the proximal end of the guidewire body. When the protective sleeve 113 is removed, the proximal connector 111 is exposed and can be inserted into the PIU 720. DETAIL B of FIG. 13B shows an example of the inner threaded section 305 of guidewire body 120 that closely matches an outer profile of the torque cable 304. In this example, the threaded section 305 has substantially triangular teeth or grooves, and drive cable 304 has a coiled wire, where the pitch of the coiled wire matches a pitch of the triangular teeth. Then, when the proximal connector 111 engages with the receptacle sleeve of the mating connector in the PIU, a pullback action causes the imaging core 300 to rotate and translate with respect to the guidewire body 120 which remains stationary.

### <Connection of Imaging Guidewire Having Dual-Drive Imaging Core to PIU>

FIG. 14 illustrates an embodiment of a PIU 720 configured to connect the imaging guidewire 100 having a pseudo rotatable imaging core 300 to the imaging console 701. According to this embodiment, the PIU 720 is substantially similar to the PIU shown and described with respect to the previous embodiment (FIG. 11). Except that in FIG. 14, the imaging core 300 includes a single fiber 302, which is rotated by the dual-drive rotation-by-translation system. Therefore, the components and functions that are similar to the previous embodiment (FIG. 11) will not be described again.

In FIG. 14, the rotation-by-translation system uses an inner threaded section 305 in the guidewire body near (proximal to) the window section 140, and a threaded guide 735 in the pullback unit 722 of PIU 720. The inner threaded section 305 in the guidewire body is configured to engage with the coiled wires of the drive cable 304, as explained above with respect to FIG. 13B. In addition, the threaded guide 735 in the pullback unit 722 engages with a threaded feature 734 provided on the outer surface of the rotary unit 724. When the PIU 720 performs an imaging pullback, the pullback unit 722 moves toward the left side of the image while causing rotation R1. The amount of rotation R1 (i.e., the number of revolutions) is limited by threaded feature 734. Since both inner threaded section 305 of the guidewire body, and threaded guide 735 in the PIU have the same features (i.e., pitch and orientation of threaded features match), the imaging core 300 is also rotated and pulled back. In this manner, the light beam emitted from the single core fiber 302 traces a helical path around the inner surface of the vessel, such that the helical path has a pitch similar to the threaded section 305 and threaded guide 735.

More specifically, similar to FIG. 11, a second gripping unit 730 engages with the tapered outer surface 112 of proximal connector 111. A first gripping unit 727 engages with the proximal end of the guidewire body 120 (i.e., with the outer hypotube), and holds the guidewire body 120 without movement, while allowing linear movement and rotation of the imaging core 300. The second gripping unit 730 includes a receiving bore portion and a centering portion 721. The receiving bore portion is configured to receive the proximal connector 111 and engage with the tared outer surface 112 of the proximal connector 111. The centering portion 721 is configured to hold the PIU-side fiber connector 729 centered and aligned with the guidewire-side proximal connector 111 of the imaging core 300.

The receiving bore of the second gripping unit 730 may have an inner surface with ridges or other structure that serves to engage with the tapered outer surface 112 of the proximal connector 111. The receiving bore of the second gripping unit 730 may preferably have a tapered surface like a Morse Taper. In this manner, when the proximal connector 111 is inserted into the PIU 720, the receiving bore of second gripping unit 730 engages with the tapered outer surface 112 and aligns the proximal connector 111 with the fiber connector 729. In addition, the second gripping unit 727 engages with the proximal end of guidewire body 120 (i.e., the outer body of the guidewire assembly), and maintains the guidewire body stationary while the imaging core 300 is simultaneously rotated by the rotary unit 724 and translated linearly by the pullback unit 722.

For this exemplary embodiment, the receiving bore of the second gripping unit 730 is directly attached to hollow-shaft motor of rotary unit 724 such that, when the proximal connector 111 is connected to the PIU 720, the inner diameter of the receiving bore (e.g., a modified telecommunications-style female connector), engages with the proximal connector 111. Precision alignment between the proximal connector 111 and the fiber connector 729 is achieved by aligning opposing conical surfaces of the two connectors. Matching conical shapes of proximal connector 111 and fiber connector 729 are held in place by the receiving bore of second gripping portion 730 and the centering portion 721, respectively, automatically centering proximal connector 111, and aligning it with the fiber connector 729. It should be kept in mind that graphical representations of optical components and angles in FIG. 14 are not to scale, and are provided herein for illustration purposes only, as it would be readily appreciated by those having ordinary skill in the art.

In operation, the FORJ of PIU 720 transmits light of an appropriate wavelength (lambda) from a source fiber 725 to a connecting fiber 726 which is terminated by a fiber connector 729. The fiber connector 729 transmits the light of wavelength lambda to the proximal connector 111, such that a single light beam LB exits from the distal optics 209 of core fiber 302. The light beam LB exits through the window section 140, and traces a helical path identical in pitch to the inner threaded section 305 of the guidewire body 120, and a pitch followed by PIU rotation & translation. In an alternate embodiment, instead of having the guidewire body 120 with an inner threaded section, it is possible to provide a molded cylinder with the inner threaded section 305; the molded cylinder can be arranged between the outer guidewire body 120 and the torque cable 304 near (proximal to) the window section 140. Likewise, a custom-engineered component such as a threaded element could replace the wires of the drive cable for optimum performance.

The single-fiber proximal connector 111 of this embodiment can be implemented using telecommunications-grade optical connectors to achieve a reliable optical connection between the reusable PIU 120 and the single-use imaging guidewire 100. In most embodiments of a single-fiber imaging core 300, LC APC type optical connectors can be used because they offer a low cost, reliable connection with minimal signal loss across the mating components of the system. The angled optical faces of the proximal connector 111 and the fiber connector 729 are polished to a high degree producing a somewhat rounded face at a precise angle to reduce back-reflection at the physical contact interface of male and female connector halves. Appropriate alignment between fiber connectors inside the PIU is important due to the need of high sensitivity necessary to collect light from the vessel—return signal from bodily tissue can be as much as 110 dB lower than excitation or input optical power. Fiber connector 729 and proximal connector 111 can be held axially aligned to each other, and also held concentric by split ceramic tubes known as mating sleeves with super fine inner surface finish, cylindricity, and diameter tolerances that enable concentricity on the order of a few microns between the input and output fibers. This embodiment can provide hi-accuracy and repeatability, which is beneficial to maintaining acceptable signal-to-noise ratio (sensitivity).

The rotate-by-translate configuration produces “limited-rotations” where the imaging core is rotated and pulled back by the PIU. The pitch of the threaded element on the core matches exactly the rotation/translation effected by the PIU, in effect, encouraging the distal end to rotate in an identical fashion as the PIU does.

FIG. 15 shows an example of an image 1500 of a conical phantom that can be acquired by the Dual-Drive PIU to guidewire connection for a single fiber configuration. The image 1500 shows a single helicoidally traced line 1512 that can vary in diameter according to conditions of the bodily lumen being imaged. For example, in regions of a vessel where there is stenosis, the diameter of the image can be reduced as shown by VD1. On the other hand, in regions of the vessel where there are branches, the helicoidally traced line can be interrupted or the diameter can have sudden increases, for example, as shown by VD2 and VD3. In a further alternate embodiment is possible to provide a dual-drive configuration applied to a multi-fiber configuration.

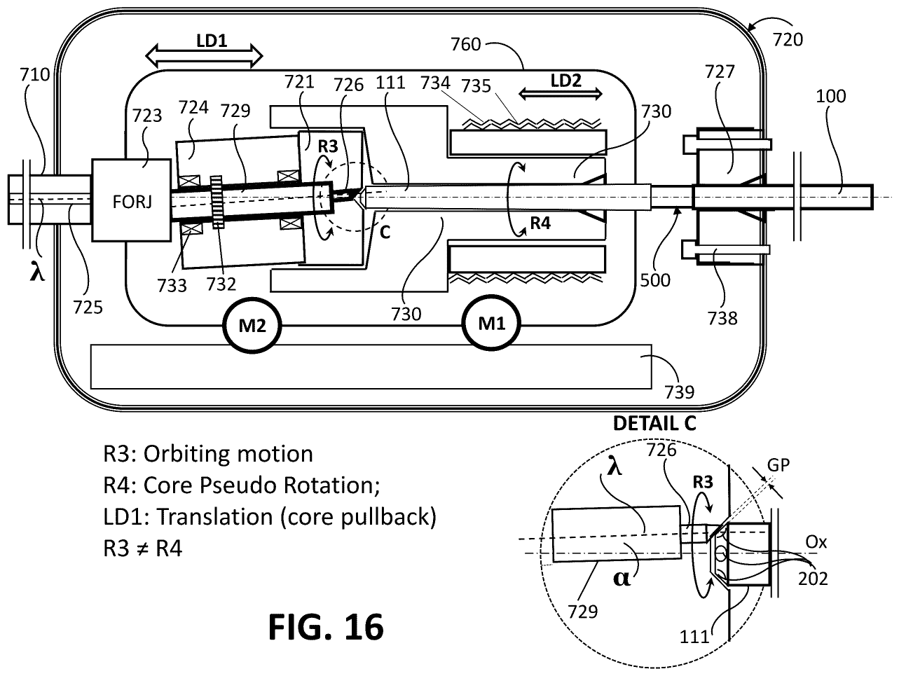

FIG. 16 illustrates an embodiment of a PIU-to-guidewire connection, where the guidewire 100 includes an imaging core 500 having a multi-fiber configuration, and the PIU controls the imaging core by a novel dual-drive rotation-by-translation system. That is, the imaging core 500 combines some features of FIG. 7C (an imaging core with multiple core fibers 202) and some features of FIG. 13 (an imaging core with a rotation-by-translation scheme). According to the embodiment of FIG. 16, the imaging core 500 includes a plurality of core optical fibers 202 similar to the first embodiment (e.g., similar to imaging core 200 shown in FIG. 4 and FIG. 7C). Proximal ends of the core optical fibers 202 are held by the proximal connector 111 at a same plane (as shown in FIG. 3 and FIG. 8A), with all fiber ends terminated with substantially the same length to be arranged on a same plane (as shown in FIG. 4B). In alternative embodiments, the distal ends of core optical fibers 202 can be staggered in a lengthwise direction (e.g., as shown in FIG. 4A or FIG. 7C). In either case, the imaging core 500 is rotated by a dual-drive rotation-by-translation system. Therefore, components and functions of the imaging guidewire 500 that are similar to the previous embodiments will not be described again.

In the embodiment of the imaging core 500 having a multi-fiber configuration rotated by a dual-drive rotation-by-translation system, the PIU-to-guidewire connection scheme is different from the previous embodiments. A source fiber 725 delivers light of one or more wavelength bands from the console 700 to the PIU 720. A fiber optic rotary joint (FORJ) 723 rotates a connecting fiber 726 and/or a fiber connector 729 so that a distal end of connecting fiber 726 fiber orbits in a path R3 with respect to the proximal connector 111. To facilitate frictionless rotation of the connecting fiber 726 with respect to proximal connector 111, there is a small gap GP between the guidewire-side fiber faces and the PIU-side optical connector 729 and/or connecting fiber 726. Light of one or more wavelengths lambda (λ) travels through the source fiber 725 arranged in the umbilical 710. The FORJ 723 transmits light received via the source fiber 725 to the connecting fiber 726, and the rotary unit 724 rotates the connecting fiber 726 and/or fiber connector 729 to orbit along path R3.

The imaging core 500 is connected to the PIU 720 by inserting the proximal connector 111 through a first gripping unit 727, a second gripping unit 730, and centering the proximal connector 111 within the centering unit 721. The first gripping unit 727 is a guidewire body grip with an inner diameter configured to hold the guidewire body 120 fixed without movement. The first gripping unit 727 can be a piece of molded plastic with through holes for attachment to the PIU 720 using bolts 738. The second gripping unit 730 is a fiber-optic component (e.g., a ferrule grip) dimensioned to grip, hold, and pullback the proximal connector 111. The second gripping unit 730 is aligned with or fixedly mounted on centering unit 721. Centering unit 721 is a precision ground component with an inner surface configured to receive and align the tapered outer surface 112 which has a Morse-like taper as shown in FIG. 8A. In this embodiment, since the rotary unit 724 does not actually rotate the imaging core 500; i.e., the PIU does not transfer torque from the FORJ to the imaging core 500 as in a conventional FORJ, the centering unit 721 does not rotate, but high-precision bearings 733 are provided to ease rotation of the connecting fiber 726 and/or fiber connector 729. The centering unit 721 has a bore with a Morse-like taper (a taper of about 6 to 12 degrees) can be sufficient for automatic collinear alignment of the proximal connector 111 with the PIU center of rotation.

To implement a pullback, the PIU 720 uses a sled-type moving platform 760 which moves linearly with respect to a base platform 739. The sled-type moving platform 760 can also be implemented as pullback unit, which as described earlier can be a translation stage with bearings or motors (e.g., M1, M2). The sled-type moving platform 760 (or pullback unit) moves in a linear direction LD1 with respect to a fixed (non-movable) base platform 739.

The rotation-by-translation system uses an inner threaded section 305 in the guidewire body near (proximal to) the window section 140, and a threaded guide 735 in the sled-type moving platform 760 of PIU 720. The inner threaded section 305 in the guidewire body is configured to engage with coiled wires of the drive cable 304, as explained above with reference to FIG. 13B, or alternatively a custom-designed component to maximize performance by adjusting pitch. In addition, the threaded guide 735 in the sled-type moving platform engages with a threaded feature 734 provided on an outer surface of the second grip unit 730. When the PIU 720 performs an imaging pullback, the pullback unit 722 moves toward the left side of the image while rotating as controlled by thread feature 734. Since both inner threaded section 305 and threaded guide 735 have the same features (same pitch and orientation match), the imaging core 500 is rotated and pulled back so that light beams traces a helical path around the inner surface of the vessel, such that the helical path from a single fiber has a pitch similar to the threaded section 305 and threaded guide 735. Note, in FIG. 16, the dual-drive threads are shown inside the sled-type moving platform 760, but these feature could be outside the sled as long as the proposed features are what cause the sled-type platform to move/translate the imaging core 500 with identical rate to that of the guidewire.

In operation, from the FORJ 723, a connecting fiber 726 arranged in a fiber connector 729 transfers light to/from the proximal connector 111. Here, the connecting fiber 726 and/or fiber connector 729 needs only orbit with respect to the proximal connector 111 and connecting fiber 726 excites the guidewire fibers sequentially as it orbits. To facilitate orbiting of the connecting fiber 726, the connecting fiber 726 is mounted on high-precision bearings 733, the connecting fiber 726 and/or fiber connector 729 are offset with respect to proximal connector 111. Here, a modified female LC connector from the telecommunications industry can be arranged slightly offset by an angle alpha (α) with respect to the longitudinal axis Ox of the guidewire body, and the connecting fiber's face is polished at an angle that precisely matches the conical angle of core fiber faces. When the guidewire 100 is attached to the PIU, a first gripping unit 727 engages with the proximal end of the guidewire body 120 (i.e., with the outer hypotube), and holds the guidewire body 120 without movement, while allowing linear movement and rotation-by-translation of the imaging core 500, in this embodiment. A second gripping unit 730 engages with the tapered outer surface 112 of the imaging core 500. The second gripping unit 730 includes a receiving shaft fixedly mounted on the sled-type moving platform 760 (pullback unit).

As shown in “DETAIL C” of FIG. 16, the end-face of connecting fiber 726 and/or the fiber connector 729 orbits in a path R3 around the proximal end of proximal connector 111, which is aligned with the central axis of the guidewire. During this orbiting movement, the connecting fiber 726 excites the guidewire core fibers through a conical surface of the proximal connector 111, such that an end face of connecting fiber 726 is held within very close proximity to end faces of core optical fibers 202 on the guidewire-side. A gap GP of 0.50 microns to 50 microns, and more preferably a gap GP of 1.0 micron to 5.0 microns, can be provided between the end face of connecting fiber 726 on the PIU-side and the end faces of core fibers 202 on the guidewire-side. This ultra-small gap GP is maintained when the proximal connector 111 of guidewire ferrule conical shape is inserted into the centering unit 721 which has conical shape to improve alignment of the proximal connector 111 with the centering unit 721. Note that the centering unit 721 does not rotate, but is coaxially aligned with first gripping unit 727 and the second gripping unit 730. The first gripping unit 727 grips the proximal end of guidewire body 120, while the second gripping unit 730 grips the outer surface 112 of the proximal connector 111. The PIU-side connecting fiber 726 orbits at up to 100 revolutions-per-second with respect to the proximal connector 111, while the non-rotating imaging core 500 is pulled back.

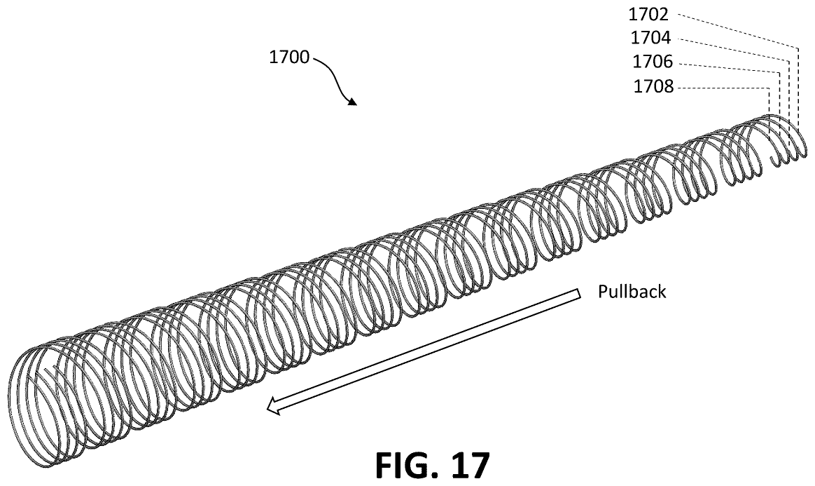

In some embodiments, a second rotation R4, referred to as a limited rotation (or pseudo rotation) of the imaging core, is controlled by the dual-drive feature (734/735), which rotates and pulls back the imaging core such that motion of imaging core 500 within the PIU matches exactly that allowed by guidewire body internal threaded features shown in FIG. 13B (DETAIL B) and FIG. 14. Hence, this is referred to a dual-drive pseudo rotation. This creates a limited core rotation R4 (rotation-by-translation) in the PIU, which matches the core rotation R2 shown FIG. 14, thereby creating an image similar to that shown in FIG. 17 (for a core with multiple fibers) or similar to image of FIG. 15 (for a single-fiber imaging core) as the connecting fiber 726 excites one core fiber 202 at a time while pulling back and rotating the core. Here, limited rotation, pseudo rotation, or limited number of revolutions refers to the rotation-by-translation effect caused by the guidewire coils following along the inner threaded section of the guidewire body. Therefore, the dual-drive rotation-by-translation concept relies namely on the pitch and angle of the wire coils. These parameters can be determined empirically based on the application and the desired level of accuracy. For a basic wireframe image, the range of rotations could be between 5-50 rotations, and preferably between 10-20 rotations. This will ensure that the imaging guidewire collects enough data to reconstruct a vessel image with sufficient accuracy, but without excessive data, which can overwhelm the user. It should also be considered that, if the pitch and angle is too large, it is possible to reduce the amount of time for imaging, but there may be a risk of missing important features from the image. Given the limited number of revolutions per pullback, it will be appreciated by those skilled in the art that light transmitted by the system travels so fast that it scans and returns from being reflected/emitted from the vessel wall tissue back into the reference arm of the interferometer almost instantaneously, certainly fast enough to enable very fast limited core rotation speeds, which will allow the system to generate a wireframe image composed of a plurality of imaging signal paths each having a helicoidally oriented path (as shown in FIG. 17).

In the embodiment shown in FIG. 16, the proximal connector 111 (a modified ceramic ferrule) is shown engaged/connected to the PIU 720. The connecting fiber 726 is enclosed in a fiber connector 729, which is rotated by a FORJ 723. A receiving unit 721 receives and aligns the proximal end of proximal connector 111, such that the distal end-face of connecting fiber 726 maintains a very small air gap with angle-polished guidewire fibers proximal end-faces. This is advantageous in keeping insertion loss and etaloning reflections to a minimum in an air gap fiber connection. The connecting fiber distal end-face is angled to closely match the polished fiber face angle on the proximal connector 111 to maintain a minimum gap. As the connecting fiber 726 scans past the guidewire core fibers, laser energy is pulsed so that light transmission is only on when the two fibers (the connecting fiber 726 and a core fiber 202) are aligned, thereby keeping reflected energy, heat, etc. to a minimum. In one embodiment, the centering unit 721 has a tapered, conical inner diameter similar to a Morse Taper, into which the proximal connector 111 (ceramic ferrule) is held as the second gripping unit 730 tightens on the ferrule. This tapered conical feature of the centering unit 721 centers the tapered, conically ground proximal end of proximal connector 111, keeping the ferrule centerline collinear with the axis of rotation within the PIU. On the PIU-side, connecting fiber 726 can be terminated in a ferrule tip which is ground and polished, and held at a precise angle to match that of the angled fiber faces of proximal connector 111, and are held very close together as the connecting fiber 726 rotates in an orbiting fashion (R3) to sequentially send and receive light to/from core optical fibers of the imaging core 500.

FIG. 17 illustrates an example of an image 1700 of a straight, tapered lumen acquired by an imaging guidewire 100 having a multi-fiber arrangement (e.g., as shown in FIGS. 4 and 7C), but controlled by a rotation-by-translation PIU-to-guidewire connection. FIG. 17 shows that image 1700 is composed of a first signal 1702 corresponding to a light beam emitted from a first fiber 202a, a second signal 1704 corresponding to a light beam emitted from a second fiber 202b, a third signal 1706 corresponding to a light beam emitted from a third fiber 202c, and a fourth signal 1708 corresponding to a light beam emitted from a fourth fiber 202d. As in previous embodiments, the diameter of the image 1700 can vary according to stenosis and/or branching of the vessel being imaged.

Advantageously, during a stenting procedure, a procedural innovation might involve initially ‘predicting’ which stent should be used based on fluoroscopy images, and then confirming that the predicted stent is actually sized correctly by imaging the stenosis with the imaging guidewire before deploying the ‘predicted’ size stent. In this case, a stent catheter is loaded onto the guidewire, mostly outside the patient's body and inside the guide catheter, and stays there while the stenosis is imaged with the imaging guidewire. If the predicted stenosis traits are unchanged after viewing the stenosis with the imaging guidewire, the pre-loaded stent catheter is advanced into place and the stent deployed. On the other hand, if the fluoroscopy images have misled a user to select the wrong size stent, the stent catheter would need to be removed so that an appropriately sized catheter can be reinserted over the imaging guidewire and into the stenosis for stent deployment. Another proposed method is to image the stenosis with a CTO-type imaging guidewire that is larger and therefore more likely to yield excellent images of the artery in question.

<Other Embodiments and/or Modifications>

The imaging guidewire disclosed herein can be employed in any noninvasive or minimal invasive medical procedure, but more particularly in intravascular imaging procedures, such as coronary imaging during angioplasty, where the risk of adverse events is minimized by the use of a novel imaging guidewire.

The materials that can be used for imaging guidewire 100 (disclosed or contemplated herein) may include those commonly associated with biocompatible medical devices. For example, the guidewire body 120 of imaging guidewire 100 and/or some of its components may be made from a metal, metal alloy, polymer, a metal-polymer composites, ceramics, or combinations thereof, and the like, or other suitable materials.

Some examples of suitable metals and metal alloys for the guidewire body 120 and the core hypotube (nested hypotubes) include, but are not limited to, stainless steel; platinum enriched stainless steel; nickel-titanium alloy (nitinol) such as linear-elastic and/or super-elastic nitinol; other nickel alloys such as nickel-chromium-molybdenum alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; or any other suitable combinations thereof.

Some examples of suitable materials for the window section include polymers or transparent metals. Window materials may comprise transparent metallic glass, liquid metal, metal glass, aerogel, polymer, monomer, polymer glass, ceramic, graphene, nano-materials, ruby, mineral, reinforced and filled polymer, acrylic, styrene, and combinations and/or equivalents thereof. Examples of polymers may include, but are not limited, to polytetrafluoroethylene (PTFE), ethylene tetrafluoroethylene (ETFE), fluorinated ethylene propylene (FEP), polyoxymethylene (POM), for example, DELRIN® available from DuPont, polyether block ester, polysterene, polyurethane (for example, Polyurethane 85A), polypropylene (PP), polyvinylchloride (PVC), elastomeric polyamides, block polyamide/ethers, polyether block amide (PEBA, for example available under the trade name PEBAX®), polyethylene terephthalate (PET), polyetheretherketone (PEEK), poly paraphenylene terephthalamide (for example, KEVLAR®). In at least some embodiments, the window section 140 can be made of transparent aluminum such as aluminum oxynitride marketed under the name ALON™ by Surmet Corp.

A novel imaging guidewire, as described herein, can be configured to obtain useful image data of an anatomical lumen such as a blood vessel, particularly a coronary artery, without the need for device swaps or with minimal device swaps. The imaging guidewire has imaging functionality similar to an MMOCT catheter, but with a size and lateral stiffness of a guidewire. A novel non-rotatable imaging core comprised of a core hypotube and a multi-fiber system allows image data collection free of NURD artifacts. Dual nested hypotubes, wherein at least one the hypotubes is a laser-cut slotted hypotube and the other one of the hypotubes telescopically moves, ensure flexibility via selective helical slotting, which varies along the length of the guidewire body. A removable, protective sleeve keeps the optical connector clean until imaging use. A modified ferrule from a standard telecommunications-grade fiber optics connector forms the proximal connector, which is also under 0.014 inches outer diameter to enable rapid exchange catheters to be back-loaded onto the guidewire body. The PIU connection system is configured to provide a two-point connection, including: a first gripping unit 727 (a guidewire body grip), and second gripping unit (a ferrule/pull grip).

Advantages of a non-rotatable imaging core include, but are not limited to, omitting rotation and therefore omitting the drive cable. This allows the imaging core to have smaller diameter, which in turn permits greater guidewire body hypotube and window wall thickness. As a result, there is less stringent material requirements for achieving adequate tensile strength and lateral stiffness, particularly in thin-walled sections. In one embodiment, the imaging core may include three to twelve core optical fibers in a sort of bolt circle arrangement around an axis. The orbiting excitation fiber on the PIU side of the connection resembles a Gatling-gun arrangement except in reverse; the core optical fibers on the guidewire-side do not rotate, but are excited sequentially in rapid succession to simulate catheter rotation while the imaging core is withdrawn. To this end, in one embodiment, the PIU includes an optical connector that rotates at an angle with respect to the longitudinal axis of the guidewire body. In this manner, the excitation fiber orbits around the proximal end of the proximal connector 111, while exciting the plurality of core fibers held by the proximal connector of the imaging core. The plurality of core optical fibers are sequentially excited by a single fiber/connector ferrule in the PIU side to create a simulated rotation. In this manner, a single interferometer is used in conjunction with a simple encoder disk, preferably attached to the rotating unit in the PIU. A computer processor is programmed to acquire and reconstruct a sort of wire-frame profile of the inner wall of a vessel with no core rotation. The use of double clad fibers can yield vessel tissue information which includes various types of plaque data that is overlaid onto the pseudo-image of the artery, further advising the clinician how to best treat the target lesion.

Another configuration that would be advantageous utilizes a concept of multiple fibers that are excited in a rapid sequential fashion to simulate core rotation to eliminate the need for accurate core/drive cable rotation. In this case, the imaging core is configured to slide in a lengthwise direction without rotation—greatly simplifying the entire OCT system and guidewire. In at least some embodiments, only the imaging core is translated linearly when imaging a lumen, the body of the guidewire that contacts the anatomy does not need to move after it has been placed in the normal manner. More specifically, image data is collected by rapidly sequenced excitation of the multiple fibers, each fiber collecting a line of ‘image’ data as the core is pulled back. The system can be configured (programed with appropriate executable code) to combine to all lines into a full 360-degree scan, and then combined into a 3D wireframe image of the vessel inner diameter (see e.g., FIG. 12, FIG. 15 and FIG. 17). Since a single interferometer is adapted to collect vessel data, the number of fibers can be scaled up by reducing the fiber size to fit more optical fibers in the inner diameter of the core hypotube until the small fiber size no longer collects enough light reflected and/or emitted from the vessel wall to function properly.

The core optical fibers arranged in the imaging core comprise distal optics configured to direct sequentially separate light beams, one light beam for each separate fiber (e.g., 2 to 48 separate light beams in one embodiment) into the wall of a vessel, and to collect light reflected, backscattered, and/or re-emitted (fluorescence) from the vessel wall through the same fiber. In one embodiment, the core optical fibers distal ends can be aligned within 100 microns or so, and are bonded in place. The core optical fibers 202 are bonded into the inner diameter of core hypotube 201. In one embodiment, the core optical fibers are also bonded into the window support tube 141. The proximal ends of the fibers are inserted and bonded into the proximal connector 111 (a ceramic ferrule), and all polished together to ensure nearly all proximal ends of the fibers are at the exact same plane (see FIG. 8A-8B) and are substantially the same length. In all of the above embodiments or combinations thereof, a computer or other signal processing device can be configured to control an electronic PIU connector (turret) to separately excite the core optical fibers 202, receive image data, and reconstruct an image by using received data.

In terms of stiffness, an imaging guidewire of the present disclosure is more stiff at the proximal end than at the distal end, with substantially constant stiffness along most of the guidewire length to properly guide catheters. The distal 10 to 20 cm of the guidewire body has a tapering stiffness towards the distal end where the distal tip terminates with a soft, atraumatic distal floppy tip. Generally related to durometer and Flexural Modulus, stiffness is greatly affected by the diameter and wall thickness of the guidewire body. In that regard, a larger profile shaft is much stiffer despite using the same durometer material. However, guidewires are severely limited in diameter profile. Therefore, in the imaging guidewire of at least one embodiment disclosed herein, there is a significant advantage in maintaining a guidewire body with a thicker wall by not using a drive cable, but using nested hypotubes with one or more non-rotating optical fibers.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

Any patent, pre-grant patent publication, or other disclosure, in whole or in part, that is said to be incorporated by reference herein is incorporated only to the extent that the incorporated materials do not conflict with standard definitions or terms, or with statements and descriptions set forth in the present disclosure. As such, and to the extent necessary, the disclosure as explicitly set forth herein supersedes any conflicting material incorporated by reference.

What is claimed is:

1. An imaging guidewire configured to be inserted into the vasculature of a patient to acquire an image of a vessel region, the imaging guidewire comprising:
a guidewire body comprised of: a first hypotube, a window made of substantially transparent material, and a flexible tip that are arranged in a lengthwise direction from a proximal end to a distal end;
an imaging core comprised of: a second hypotube, one or more core optical fibers arranged along an inner surface of the second hypotube, and a proximal connector attached to a proximal end of the second hypotube,
wherein the proximal connector is configured to connect the imaging core to a patient interface unit (PIU),
wherein the second hypotube is nested inside the first hypotube, such that to acquire an image of the vessel region, the second hypotube is pulled back and pseudo rotated, or only pulled back, with respect to the first hypotube, while the one or more core optical fibers scan the vessel region with light of one or more wavelengths transmitted through the window.

2. The imaging guidewire according to claim 1,
wherein the each one of the one or more core optical fibers includes a distal optics assembly at the distal end thereof, and
wherein the distal optics assembly of each one of the one or more core optical fibers includes a focusing component and a beam directing component configured to collectively transmit a light beam towards the vessel region, and collect light reflected, scattered, and/or emitted by the vessel region, and further configured to guide the collected light through the same one or more core optical fibers back to the proximal connector.

3. The imaging guidewire according to claim 1,
wherein the guidewire body includes a rigid section at the proximal end of the first hypotube and a semi-rigid section at the distal end of the first hypotube, and
wherein a portion of the semi-rigid section is embedded into the material of the window.

4. The imaging guidewire according to claim 3,
wherein the semi-rigid section includes laser-cut helicoidally-oriented coils, and wherein the laser-cut helicoidally-oriented coils have a variable pitch and a variable cross-section.

5. The imaging guidewire according to claim 4,
wherein the semi-rigid section is embedded into the material of the window such that the laser-cut helicoidally-oriented coils are progressively etched, and have an increasingly shorter pitch and an increasingly smaller cross-section in a direction from the proximal end towards the distal end, so that the most-distal coils are substantially encapsulated within the material of the window.

6. The imaging guidewire according to claim 4,
wherein the semi-rigid slotted section is embedded into the material of the window such that the laser-cut helicoidally-oriented coils have an increasingly shorter pitch and an increasingly smaller cross-section in a direction from the proximal end towards the distal end so that the laser-cut helicoidally-oriented coils at the distal end of the semi-rigid slotted section are partially embedded within the material of window and covered by a thin heat-shrinkable tube to simulate encapsulation.

7. The imaging guidewire according to claim 1,
wherein the flexible tip includes a coiled wire embedded in the material of the window, an atraumatic cap formed at the distal end of the coiled wire, and a core wire attached to the atraumatic cap, and
wherein the core wire extends proximally from the atraumatic cap to the window.

8. The imaging guidewire according to claim 7, further comprising:
a window support tube arranged along an inner surface of the window and extending to the flexible tip,
wherein the flexible tip is integrally coupled to the window via the coiled wires which are embedded into the material of the window in a direction from the distal end towards the proximal end, and
wherein the core wire extends proximally from the atraumatic cap to a boubous element which is configured to engage with the window support tube so as to prevent the flexible tip from becoming disconnected from the guidewire body.

9. The imaging guidewire according to claim 1,
wherein the second hypotube has an outer diameter smaller than an inner diameter of the first hypotube, and
wherein the one or more core optical fibers includes a plurality of core optical fibers fixedly held within an inner surface of the second hypotube.

10. The imaging guidewire according to claim 9,
wherein the proximal connector holds proximal ends of the plurality of core optical fibers at a same proximal plane and arranged substantially parallel to, and evenly distributed around, a central axis of the second hypotube, and all of core optical fibers have substantially the same length, such that distal ends of all core optical fibers are aligned with the window at a same distal plane.

11. The imaging guidewire according to claim 9,
wherein the proximal connector holds proximal ends of the plurality of core optical fibers at a same proximal plane and arranged substantially parallel to, and evenly distributed around, a central axis of the second hypotube, and each of the core optical fibers has a different length, such that distal ends of all core optical fibers are staggered in the lengthwise direction such that a distal end of each core optical fiber is at a different distal plane with respect to the window.

12. The imaging guidewire according to claim 10,
wherein the plurality of core optical fibers are configured to receive the light from a single excitation optical fiber arranged in the PIU,
wherein each core optical fiber of the imaging core transmits the light from a proximal end to a distal end thereof, and
wherein the plurality of core optical fibers irradiate the vessel region through the window with a corresponding plurality of light beams emitted from distal ends of the plurality of optical fibers.

13. The imaging guidewire according to claim 12,
wherein the distal ends of the plurality of optical fibers are configured to collect light reflected, backscattered, and/or emitted from the vessel region, and
wherein the plurality of core optical fibers transmit the collected light back to the PIU independently through each core optical fiber.

14. The imaging guidewire according to claim 10,
wherein the proximal connector is a modified fiber connector configured to hold the plurality of core optical fibers in a non-rotatable manner within an inner diameter of the second hypotube.

15. The imaging guidewire according to claim 10,
wherein the plurality of core optical fibers output a corresponding plurality of light beams through the window in a pseudo rotatable manner without rotating the first hypotube.

16. The imaging guidewire according to claim 10,
wherein the proximal connector is a modified fiber-optic ferrule having a plurality of micro-channels that hold the plurality of core optical fibers evenly distributed around a central axis of the proximal connector, and
wherein the plurality of core optical fibers receive the light from an orbiting optical fiber in a reverse Gatling-gun arrangement such that the core optical fibers irradiate the vessel region by rapidly sequenced excitation of the plurality of core optical fibers.

17. The imaging guidewire according to claim 10,
wherein distal ends of the plurality of core optical fibers are staggered in a lengthwise direction with respect to the window, and
wherein the distal ends of the plurality of core optical fibers are configured to output a corresponding plurality of light beams at different angles with respect to a longitudinal axis of the second hypotube, such that each core optical fiber scans a different sector of the vessel region.

18. The imaging guidewire according to claim 1,
wherein the proximal connector has a conical outer surface that tapers in a direction from the proximal end towards the distal end, and
wherein, when the imaging guidewire is connected to the PIU, the conical outer surface is configured to facilitate insertion and precise alignment of the proximal connector with a receiving conical bore of the PIU.

19. The imaging guidewire according to claim 1, further comprising:
a drive cable made of wire that is helicoidally coiled at a predetermined pitch, wherein the drive cable is arranged in the imaging core fixedly attached to the second hypotube, and
wherein the first hypotube includes an inner threaded section having helicoidally oriented grooves that matches an outer profile of helicoidally coiled wire of the drive cable.

20. The imaging guidewire according to claim 19,
wherein the one or more core optical fibers includes a single core optical fiber, and wherein the proximal connector is a modified fiber ferrule that holds a proximal end of the single core optical fiber arranged substantially concentric with a central axis of the second hypotube.

21. The imaging guidewire according to claim 20,
wherein the second hypotube holds the single core optical fiber, such that a distal end of the single core optical fiber is aligned with the window,
wherein the proximal connector receives light from a non-rotating optical fiber arranged in a pullback unit of the PIU, and receives a mechanical force from the pullback unit to telescopically translate the imaging core with respect to the guidewire body,
wherein, when the imaging core is telescopically translated with respect to the guidewire body, the inner threaded section of the first hypotube that matches the outer profile of helicoidally coiled wire of the drive cable causes the imaging core to simultaneously translate and pseudo rotate with respect to the guidewire body, and
wherein pullback unit of the PIU drives limited rotation in identical fashion as that caused by distal threaded inner diameter of first hypotube.

22. The imaging guidewire according to claim 21,
wherein the single core optical fiber scans the vessel region through the window with a single beam of light emitted from the distal end of the single core optical fiber, while the imaging core is simultaneously telescopically translated and pseudo rotated, while the guidewire body remains stationary.

23. The imaging guidewire according to claim 19,
wherein the imaging core includes a plurality of core optical fibers, and
wherein the proximal connector is a ceramic ferrule that holds proximal ends of the plurality of core optical fibers arranged substantially equidistant from a central axis of the proximal connector.

24. The imaging guidewire according to claim 23,
wherein the second hypotube holds distal ends of the plurality of core optical fibers aligned with the window,
wherein the proximal connector receives light from an orbiting optical fiber arranged in a rotating unit of the PIU, and receives a mechanical force from a pullback unit arranged in the PIU to telescopically translate the imaging core with respect to the guidewire body, and
wherein, when the imaging core is telescopically translated and rotated with respect to the guidewire body through a limited number of revolutions corresponding to the length of the inner threaded element or segment of the guidewire body inner diameter, the inner threaded section that closely matches the outer profile of helicoidally coiled wire of the drive cable or custom threaded element, causes the imaging core to simultaneously translate and pseudo rotate through a limited number of revolutions with respect to the guidewire body.
